(19) United States
(12) Patent Application Publication
(10) Pub. No.: US 2004/0185453 A1
(43) Pub. Date: Sep. 23, 2004

(54) Affinity based methods for separating homologous parental genetic material and uses thereof

(76) Inventors: Joel Myerson, Berkeley, CA (US); Jeffrey R. Sampson, San Francisco, CA (US); Michael Thomas Barrett, Mountain View, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/394,915

(22) Filed: Mar. 21, 2003

(51) Int. Cl. C12Q 1/68; C07H 21/04

(52) U.S. Cl. 435/6; 536/25.4

(57) ABSTRACT

The present invention provides general affinity based methods for separating parental genetic material. Without limitation, the inventive methods may be used to separate maternal genetic material from paternal genetic material for haplotyping purposes. According to such embodiments, once the maternal genetic material has been separated from the paternal genetic material any method of SNP genotyping can be used, and the SNP genotypes will be, by definition the genetic haplotypes.

### BACKGROUND OF THE INVENTION

Single nucleotide polymorphisms (SNPs) are single nucleotide sites that exist in two to four variations in the genetic material of an interbreeding population, e.g., the human genome (Sunyaev, Trends in Genetics 16:335, 2000). To date, nearly 3 million putative SNPs scattered throughout the human genome of 3 billion base pairs (bp) have been deposited into public databases. This corresponds to approximately one SNP for every 1,000 bp in the genome. A small fraction of this genetic variation is likely to explain the majority of the differences between individuals, including differences in their levels of response to defined forms of treatment and differences in their predisposition to development of many common human diseases, e.g., cardiovascular disease, hypertension, diabetes, asthma, neurological disease, cancer, etc. For these reasons, an increasing number of SNPs are being used as genetic markers for biomedical research and clinical diagnostics.

Moreover, it is becoming increasingly clear, that genetic “haplotypes”, which are defined as the identity of a collection of SNPs as they reside on either the maternal or the paternal genetic material of an individual (e.g., without limitation a maternal chromosome or part thereof), have much greater information content than the identity of individual SNPs. Haplotypes exist because groups of SNPs tend to be linked within chromosomes. As a consequence of linkage disequilibrium, only a small number of common haplotypes are generally found in a specific population (e.g., haplotypes that span as much as 100,000 bp have been shown to exist in just a few different versions). Furthermore, in any one individual, only two versions of a particular haplotype exist (i.e., one inherited from each parent). Accordingly, the use of haplotypes promises to dramatically reduce the complexity of genetic analysis, e.g., when making associations between SNPs and complex diseases.

In addition, haplotypes are thought to predict the activity of genes more precisely than individual SNPs. This is because individual polymorphisms may have different effects on the biological function of a gene. A haplotype integrates these different, sometimes opposing, effects into a single piece of information, the sum of the effects of the collection of SNPs. Thus a single polymorphism that, in isolation, might have had a slightly negative effect on gene function, would be associated with increased activity when present in a haplotype where the net effect of all the polymorphisms was positive. For these reasons, determining the haplotype of individuals will likely become standard practice.

In order to perform haplotyping, it is generally necessary to obtain, either directly or indirectly, information about the identities of a collection of SNPs on a single parental chromosome or chromosome fragment.

Indirect methods typically involve using haplotype based association tests (Service et al., Am. J. Hum. Genet. 64:1728, 1999 and Akey et al., Eur. J. Gen. 9:291, 2001). For example, parental genotyping can be used to infer haplotypes in a family study, although in many cases it is impractical or impossible to obtain genetic material from parents (Hodge and Boehnke, Nat. Genet. 21:360, 1999).

The haplotype of a genomic sample can also be determined directly using mass spectrometry methods if the SNPs are close enough together to be detectable on the same molecule (Griffin and Smith, Trends Biotechnol. 18:77-84, 2000). However, even for closely spaced SNP combinations, there will exist cases where the haplotypes form a degenerate mixture (as determined by mass) that will not be distinguishable using current mass spectroscopic methods. For example, while: -A-G- can be distinguished from -A-T-, -A-G- cannot be distinguished from -G-A-.

Currently, most direct methods for haplotyping multiple SNP sites that are separated by large distances require the initial separation of the maternal and paternal genetic material by cloning followed by SNP genotyping of the separated material. Typically, individual chromosomes are cloned using either the Clasper vector system (Bradshaw et al., Nucleic Acids Res. 23:4850, 1995) or the transformation-associated recombination (TAR) cloning vector method (Kouprina et al., Proc. Natl. Acad. Sci. USA 95:4469, 1998). Using these methods, chromosome fragments ranging in size from 1,000 to 300,000 bp can be cloned, isolated, and subsequently SNP genotyped. However, a number of limitations are associated with current cloning methods. These limitations include, for example, the difficulty of generating a set of clones representing entire chromosomes, errors introduced during the cloning process, and the time required to perform what is a very complex and multi-step process.

### SUMMARY OF THE INVENTION

The present invention provides general affinity based methods for separating homologous parental genetic material. Without limitation, the inventive methods may be used to separate maternal genetic material from homologous paternal genetic material for haplotyping purposes. According to such embodiments, once the maternal and paternal homologues have been separated from each other any method of SNP genotyping can be used, and the SNP genotypes will be, by definition the genetic haplotypes.

According to the instant invention, homologous components of the parental genetic material are physically separated by virtue of their differential affinities for one or more binding entities. In general, the differential affinities are based on the presence of different alleles in the maternal and paternal genetic material. For example, without limitation, an inventive binding entity may include a molecular probe that binds preferentially to an allele in the maternal genetic material over its heterozygous counterpart in the paternal genetic material (or vice versa). As will be described in greater detail below, each binding entity may include one or more molecular probes that include a polypeptide, a small molecule, an oligonucleotide, or a combination thereof.

### DESCRIPTION OF THE DRAWING

FIG. 1 depicts the binding between a binding entity and parental genetic material. In the illustrated embodiment, the binding entity includes an oligonucleotide associated with a solid phase. The oligonucleotide is complementary to one of the strands of the double stranded genetic material.

FIG. 2 depicts the binding between a binding entity and parental genetic material. In the illustrated embodiment, the binding entity includes two different oligonucleotides associated with a solid phase. The two different oligonucleotides are complementary to the two strands of the double stranded genetic material.

FIG. 3 depicts the binding between a binding entity and parental genetic material. In the illustrated embodiment, the binding entity includes an oligonucleotide associated with a solid phase. The oligonucleotide forms a hairpin loop structure that is complementary to each of the two strands of the double stranded genetic material.

FIG. 4 depicts the binding between a binding entity and parental genetic material. In the illustrated embodiment, the binding entity includes an oligonucleotide associated with a solid phase. The oligonucleotide includes an unstructured nucleic acid that is complementary to each of the two strands of the double stranded genetic material.

FIG. 5 depicts a sequential process for separating maternal and paternal genetic material. In the illustrated embodiment, the maternal and paternal genetic material are homozygous AA, bb, CC, and EE for four polymorphic regions and heterozygous dD for a fifth polymorphic region.

FIG. 6 depicts an embodiment of a system that uses a sequential separation process to separate maternal and paternal genetic material.

FIGS. 7A and 7B depict an operational embodiment of the system that is depicted in FIG. 6.

### DEFINITIONS

“Allele”: As defined herein, “alleles” of a polymorphic region are mutually exclusive versions of a polymorphic region. For example, without limitation, a polymorphic region that includes a single SNP site can potentially exist as one of four different alleles, namely: -A-, -C-, -T-, and -G-.

“Associated with”: When two entities are “associated with” one another as described herein, they are linked by direct or indirect covalent or non-covalent interactions. Indirect interactions might involve a third entity that is itself associated with both the first and second entities. Desirable non-covalent interactions include, for example, ionic interactions, hydrogen bonds, van der Walls interactions, hydrophobic interactions, etc. In certain embodiments, the non-covalent interactions are ligand/receptor type interactions. Any ligand/receptor pair with a sufficient stability and specificity to operate in the context of the invention may be employed to associate two entities. To give but an example, a first entity may be covalently linked with biotin and a second entity with avidin. The strong non-covalent binding of biotin to avidin would then allow for association of the first entity with the second entity. Typical ligand/receptor pairs include antibody/antigen, protein/co-factor, and enzyme/substrate pairs. Besides the commonly used biotin/avidin pair, these include without limitation, biotin/streptavidin, digoxigenin/anti-digoxigenin, FK506/FK506-binding protein (FKBP), rapamycin/FKBP, cyclophilin/cyclosporin, and glutathione/glutathione transferase pairs. Other suitable ligand/receptor pairs would be recognized by those skilled in the art, e.g., monoclonal antibodies paired with a epitope tag such as, without limitation, glutathione-S-transferase (GST), c-myc, FLAG®, and maltose binding protein (MBP) and further those described in Kessler pp. 105-152 of Advances in Mutagenesis” Ed. by Kessler, Springer-Verlag, 1990; “Affinity Chromatography: Methods and Protocols (Methods in Molecular Biology)” Ed. by Pascal Baillon, Humana Press, 2000; and “Immobilized Affinity Ligand Techniques” by Hermanson et al., Academic Press, 1992. Phenylboronic acid complexes may also be used for preparing affinity tag/capture agent pairs (e.g., as described in U.S. Pat. No. 5,594,151). In addition, polyA/polyT pairs may be used.

“Binds preferentially”: As described herein, when a molecular probe “binds preferentially” to a first allele over a second allele (e.g., that differ by a single SNP variation), the molecular probe is able to discriminate between genetic material that includes the different alleles. In particular, it is to be understood that under those conditions, the molecular probe can be used to separate genetic material that includes the different alleles. In certain non-limiting embodiments, a molecular probe is said to “bind preferentially” to a first allele over a second allele when the binding affinity of the molecular probe for parental genetic material that includes the first allele is a factor of 2, 5, 10, 20, 50, 100, or more greater than for a parental homologue that includes the second allele.

“Haplotype”: The term “haplotype”, as used herein, refers to the identity of a collection of two or more SNPs as they reside on either one of a homologous pair of chromosomes. For the purposes of the present invention, the collection may span an entire chromosome or parts of a chromosome.

“Heterozygous”: As defined herein, the maternal and paternal genetic material of an individual is “heterozygous” for a particular polymorphic region when the maternal and paternal genetic material include different alleles of that polymorphic region.

“Homozygous”: As defined herein, the maternal and paternal genetic material of an individual is “homozygous” for a particular polymorphic region when the maternal and paternal genetic material include the same allele of that polymorphic region.

“Homologous”: As defined herein, “homologous” maternal and paternal genetic material refers to any form of genetic material that has been derived from a pair of homologous chromosomes. Typically, homologous chromosomes have approximately the same length, centromere position, visible structure, staining pattern, pair during meiosis, and are similar with respect to their constituent genetic loci. Pairs of homologous chromosomes are generally homozygous for certain polymorphic regions and heterozygous for others. It is to be understood that this definition encompasses full length chromosomes and chromosome fragments, e.g., those generated by mechanical protocols such as shearing and sonication; chemical protocols such as enzymatic digestion, polymerase extension, etc. In addition, it is to be understood that this definition also encompasses polynucleotide molecules, e.g., without limitation, cDNA molecules, plasmids, and PCR products that have been derived from homologous chromosomes or fragments thereof, e.g., by cloning (e.g., see Bradshaw et al., Nucleic Acids Res. 23:4850, 1995 and Kouprina et al., Proc. Natl. Acad. Sci. USA 95:4469, 1998); whole genome amplification (WGA, e.g., see Zheng et al., Cancer Epidemiol. Biomarkers Prev. 10:697, 2001); multiple displacement amplification (MDA, e.g., see Dean et al., Proc. Natl. Acad. Sci. USA 99:5216, 2002); etc.

“Modified bases”: Modified bases, as defined herein, are bases having a structure derived from the naturally occurring bases adenine (A), thymine (T), guanine (G), cytosine (C), and uracil (U). For example, without limitation a modified adenine base has a structure comprising at least a purine with a nitrogen atom covalently bonded to C6 of the purine ring as numbered by conventional nomenclature known in the art. In addition, it is recognized that modifications to the purine ring and/or the C6 nitrogen may also be included in a modified adenine. A modified guanine base has a structure comprising at least a purine, and an oxygen atom covalently bonded to the C6 carbon. Modifications to the purine ring and/or the C6 oxygen atom may also be included in modified guanine bases. A modified cytosine base has a structure comprising at least a pyrimidine and a nitrogen atom covalently bonded to the C4 carbon as numbered by conventional nomenclature known in the art. Modifications to the pyrimidine ring and/or the C4 nitrogen atom may also be included in modified cytosine bases. A modified thymine base has a structure comprising at least a pyrimidine, an oxygen atom covalently bonded to the C4 carbon, and a C5 methyl group. Again, it is recognized by those skilled in the art that modifications to the pyrimidine ring, the C4 oxygen and/or the C5 methyl group may also be included in a modified thymine. A modified uracil base may have a structure comprising at least a pyrimidine, an oxygen atom covalently bonded to the C4 carbon and a C5 hydrogen. Modifications to the pyrimidine ring and/or the C4 oxygen may also be included in a modified thymine. Some non-limiting examples of modified bases include 2-aminoadenine, 2-thiothymine, 3-methyladenine, 5-propynylcytosine, 5 -propynyluracil, 5-bromouracil, 5-fluorouracil, 5-iodouracil, 5-methylcytosine, 7-deazaadenine, 7-deazaguanine, 8-oxoadenine, 8-oxoguanine, O(6)-methylguanine, and 2-thiocytosine.

“Modified oligonucleotide”: A modified oligonucleotide, as defined herein, is an oligonucleotide having a modification to its chemical structure. Oligonucleotides that include modified sugars (e.g., 2′-fluororibose, arabinose, hexose, and riboses with a 2′-0, 4′-C-methylene bridge), modified bases, and/or modified phosphate groups (e.g., phosphorothioates and 5′-N-phosphoramidite linkages) are considered modified oligonucleotides as defined herein. Without limitation, modified oligonucleotides disclosed herein include peptide nucleic acids (PNAs), locked nucleic acid (LNAs), and unstructured nucleic acids (UNAs).

“Naturally occurring bases”: Naturally occurring bases are defined for the purposes of the present invention as adenine (A), thymine (T), guanine (G), cytosine (C), and uracil (U). It is recognized that certain modifications of these bases occur in nature. However, for the purposes of the present invention, modifications of A, T, G, C, and U that occur in nature are considered to be non-naturally occurring. For example, 2-aminoadenine is found in nature, but is not a “naturally occurring base” as that term is used herein. Other non-limiting examples of modified bases that occur in nature but are considered to be non-naturally occurring herein are 5-methylcytosine, 3-methyladenine, O(6)-methylguanine, and 8-oxoguanine.

“Nucleic acid sequence”: The “nucleic acid sequence” or “sequence” of a polynucleotide is defined by the sequential identity of the bases of the nucleotides in the polynucleotide molecule. The sequence of a polynucleotide is read from the 5′ to the 3′ end of the chain.

“Polymorphic region”: A “polymorphic region” as defined herein is a region of genetic material that includes one or more SNP sites, e.g., 1, 2, 3, 4, 5, or more sites. It is to be understood that polymorphic regions may be located in protein coding regions (e.g., exons) or non-coding regions (e.g., introns, promoter and gene regulatory regions, origins of replication, telomeres, and non-functional intergenic DNA regions) of the genetic material. As defined herein, polymorphic regions span at least 6 nucleotides and include at least one SNP site. In certain embodiments, a polymorphic region may include 7, 8, 9, 10, 11, 12, 13, 14, 15, 16, 17, 18, 19, 20, or more nucleotides.

“Polynucleotide”, “nucleic acid”, or “oligonucleotide”: The terms “polynucleotide”, “nucleic acid”, or “oligonucleotide”, as used herein, refer to a polymer of nucleotides. The terms “polynucleotide”, “nucleic acid”, and “oligonucleotide”, may be used interchangeably. Typically, a polynucleotide comprises at least two nucleotides linked together by phosphodiester bonds. DNA and RNA are exemplary oligonucleotides of the present invention. In general, the polynucleotides may be single stranded or double stranded. In certain embodiments, the polynucleotides may contain naturally occurring nucleotides (i.e., nucleotides that include the bases adenine, thymine, cytosine, guanine, or uracil). In certain embodiments, the polynucleotides may include modified nucleotides (e.g., without limitation, nucleotides that include the bases 2-aminoadenine, 2-thiothymine, 3-methyladenine, 5-propynylcytosine, 5-propynyluracil, 5-bromouracil, 5-fluorouracil, 5-iodouracil, 5-methylcytosine, 7-deazaadenine, 7-deazaguanine, 8-oxoadenine, 8-oxoguanine, O(6)-methylguanine, or 2-thiocytosine). Alternatively or additionally, the oligonucleotides may include modified sugars (e.g., 2′-fluororibose, arabinose, hexose, and riboses with a 2′-O, 4′-C-methylene bridge) and/or modified phosphate groups (e.g., phosphorothioates and 5 ′-N-phosphoramidite linkages). Without limitation, the present invention encompasses the use of biomolecules that include peptide nucleic acids (PNAs), locked nucleic acid (LNAs), and unstructured nucleic acids (UNAs). Also, one or more of the nucleotides in an inventive polynucleotide may be modified, for example, by the addition of a chemical entity such as a linker for conjugation, functionalization, or other modification, etc.

“Population”: The term “population”, as used herein, refers to human as well as non-human populations, including, for example, populations of mammals, birds, reptiles, amphibians, and fish. Preferably, the non-humans are mammals (e.g., rodents, mice, rats, rabbits, monkeys, dogs, cats, primates, or pigs).

“Protein”, “polypeptide”, or “peptide”: The terms “protein”, “polypeptide”, and “peptide” refer to a polymer of amino acids. The terms “protein”, “polypeptide”, and “peptide”, may be used interchangeably. Typically a polypeptide includes a string of at least two amino acids linked together by peptide bonds. Inventive proteins may contain naturally occurring amino acids and non-naturally occurring amino acids (i.e., amino acids that do not occur in nature but that can be incorporated into a polypeptide chain). Also, one or more of the amino acids in an inventive polypeptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Single nucleotide polymorphism”: The term “single nucleotide polymorphism”, as used herein, refers to single nucleotide sites that exist in two to four variations within the genetic material of an interbreeding population, e.g., within the human genome. The terms “single nucleotide polymorphism”, “polymorphism”, and “SNP”may be used interchangeably.

### DETAILED DESCRIPTION OF CERTAIN PREFERRED EMBODIMENTS OF THE INVENTION

This patent application mentions various patents, patent applications, and published references. The contents of each such item are hereby incorporated by reference.

The present invention provides general affinity based methods for separating homologous parental genetic material. Without limitation, the inventive methods may be used to separate maternal genetic material from homologous paternal genetic material for haplotyping purposes. According to such embodiments, once the maternal genetic material and the paternal homologue have been separated from each other any method of SNP genotyping can be used, and the SNP genotypes will be, by definition the genetic haplotypes.

According to the instant invention, homologous components of the parental genetic material are physically separated by virtue of their differential affinities for one or more binding entities. In general, the differential affinities are based on the presence of different alleles in the maternal and paternal genetic material. For example, without limitation, an inventive binding entity may include a molecular probe that binds preferentially to an allele in the maternal genetic material over its heterozygous counterpart in the paternal genetic material (or vice versa).

Each binding entity may include one or more molecular probes that include a polypeptide, a small molecule, an oligonucleotide, or a combination thereof. Certain exemplary embodiments of these molecular probes are described in greater detail below. However, it is to be understood that the methods and devices of the present invention are in no way limited to these specific molecular probes and that any molecular probe that binds preferentially to an allele of interest may be used.

1. Molecular Probes

In general, the molecular probes of the present invention are designed to bind preferentially to a particular allele. As defined above, an allele is a specific version of a polymorphic region and includes a particular variation of at least one SNP site. Again, as defined above, a polymorphic region spans at least 6 nucleotides.

Although not required, in certain embodiments, the size of the allele that is preferentially bound by a molecular probe may be selected to provide a unique address within the genetic material of interest.

To illustrate this, consider for example a 13 kb DNA molecule. If one assumes that the four naturally occurring DNA bases (i.e., A, T, G, and C) are randomly distributed within the sequence of the DNA molecule, then statistically, a particular sequence of 6 nucleotides (e.g., ATTGCT) should only occur once in every 4096 (i.e., 4⁶) nucleotides. Statistically, the sequence of 6 nucleotides would therefore be expected to occur a total of about three times within the 13 kb sequence. In order to obtain a unique address within the 13 kb sequence, one would therefore need to choose a sequence of at least 7 nucleotides. More generally, in order to provide a unique address within a polynucleotide that includes a total of X nucleotides, one would need to choose a sequence with a length Y that equals at least the smallest integer value that is greater than or equal to the natural logarithm of X divided by the natural logarithm of 4 (i.e., Y=at least smallest integer value that is ≧In X/In 4≈In X/1.38629436). In certain embodiments, the length of the allele sequence is chosen to include 5, 4, 3, 2, or 1 nucleotide(s) more than is required by the above formula.

The human genome includes about 3 billion base pairs. Accordingly, based on the above assumptions, providing a unique address within the entire genome would require a sequence of at least 16 nucleotides. Individual chromosomes range in size from 50 to 250 million base pairs, accordingly, providing a unique address within individual chromosomes may require a sequence of between about 13 and 14 nucleotides. Chromosome fragments can range anywhere from a 1,000 bases upwards, accordingly providing a unique address within a chromosome fragment may require as little as a sequence of about 6 nucleotides.

Molecular Probes That Include a Polypeptide

In certain embodiments of the present invention, an inventive binding entity may include a molecular probe that includes a polypeptide.

In preferred embodiments, the polypeptide includes a DNA-binding motif, preferably a sequence specific DNA-binding motif. A variety of sequence specific DNA-binding motifs have been described in the art, e.g., those found in prokaryotic repressors and activators, or eukaryotic transcription factors, nucleases, and polymerases (Struhl, Annu. Rev. Biochem. 58:1051, 1989; Johnson and McKnight, Annu. Rev. Biochem. 58:799, 1989; Pabo and Sauer, Annu. Rev. Biochem. 61:1053, 1992; Gehring et al., Annu. Rev. Biochem. 63:487, 1994; and Suzuki et al., Protein Engineering, 8:329, 1995). These include, for example, helix-turn-helix motifs and zinc-finger motifs.

The most common DNA-binding motif is the helix-turn-helix motif. This motif consists of two α-helices that are held at a fixed angle and connected by an extended α-turn chain of amino acids. The COOH-terminal, or recognition helix, fits into the major groove of DNA, an interaction that is modulated by amino acid residues at the outer helical surface and by the conformation of peptides that house the domain. The helix-turn-helix conformation is a component of the homeobox, a conserved domain of about 60 amino acids within D. Melanogaster homeotic gene products. This homeobox domain has also been identified in many invertebrate and vertebrate regulators of gene expression (Latchman in “Eukaryotic transcription factors”, Academic Press, London, 1999). Other well-studied examples of helix-turn-helix motif proteins are Lac repressor, 434 cro, 434 repressor, Trp repressor and LexA (Harrison and Aggarwal, Annu. Rev. Biochem. 59:933, 1990).

Zinc-finger motifs recruit zinc in order to bind DNA. Three different families of zinc-finger motifs have been identified (Berg, Annu. Rev. Biophys. Biophys. Chem. 19:405, 1990 and Berg, Proc. Natl. Acad. Sci. USA 85:99, 1988). The “classic” zinc-finger motif consists of about 30 amino acids and includes two invariably positioned cysteine-histidine pairings that co-ordinate tetrahedral binding to a single zinc atom (Miller et al., EMBO J. 4:1609-1614, 1985). The DNA-binding region of the hormone-receptor family of transcription factors includes zinc-finger motifs with four cysteines that co-ordinate to a single zinc atom. Retroviral DNA-binding proteins contain zinc finger motifs with about 18 amino acids in which one zinc atom is bound to three cysteines and one histidine residue in the order Cys-Cys-His-Cys. Specific amino acids within the zinc-finger motif interact in a sequence-specific manner with three adjacent base pairs of the double-stranded DNA (or in some case RNA) (Pavletich and Pabo, Science 252:809, 1991). The SP1 family of transcription factors include three linked zinc-finger motifs and can recognize up to nine contiguous base pairs.

Over the past several years, much effort has been focused on understanding the rules that govern the recognition specificity of zinc-finger motifs with the goal of engineering DNA-binding proteins that bind defined DNA sequences of various lengths with high specificity (see PCT Publication No. WO 00/42219; Desjarlais and Berg, Proc. Natl. Acad. Sci. USA 89:7345, 1992; Rebar and Pabo, Science 263:671, 1994; Nagaoka and Sugiura, J. Inorganic Biochem. 82:57, 2000; Dreier et al., J. Mol. Biol. 303:489, 2000; Greisman and Pabo, Science 275:657, 1997; and Rebar et al., Methods Enzymol. 267:129, 1996). Importantly, polypeptides that include zinc-finger motifs can be designed to have strong affinities for their cognate DNA-binding site exhibiting apparent binding constants (K_{d}) in the low to sub-nanomolar range with specificity constants, i.e., K_{d}(cognate)/K_{d}(non-cognate) favoring the cognate DNA by about 100 fold. These properties enable polypeptides with zinc-finger motifs to bind DNA samples of high sequence complexity (e.g., human genomic DNA) with high specificity.

A particular advantage of using zinc-finger motifs in polypeptides of the present invention stems from the fact that binding affinities are sensitive to the concentration of free Zn²⁺ in the medium and hence can be adjusted by adding or removing a zinc chelator such as EDTA (Frankel et al., Proc. Natl. Acad. Sci. USA 84:4841, 1987).

Molecular Probes That Include a Small Molecule

In other embodiments of the present invention, an inventive binding entity may include a molecular probe that includes a small molecule.

The small molecule may, for example, include an inorganic complex that intercalates into the major groove of DNA, e.g., a 9,10-phenanthrenequinone diimine complex of rhodium (III), a 9,10-phenanthrenequinone diimine 2,2′-bipyridyl complex of rhodium (III), or a derivative thereof described in Sitlani and Barton, Biochemistry, 33:12100, 1994.

Alternatively, the molecular probe may include a polyamide that contains imidazole and pyrrole amino acids. In particular, preferred the polyamides are prepared from N-methylpyrrole, N-methylimidazole, and N-methyl-3-hydroxypyrrole amino acids (White et al., Nature 391:468, 1998). For example, the natural product netropsin contains two N-methylpyrrole units and forms a 1:1 complex in the minor groove of DNA with adenine and thymine-rich DNA fragments (Kopka et al., Proc. Natl. Acad. Sci. USA 82:1376, 1985 and Lown et al., Biochemistry 25:7408, 1986). Distamycin, also a natural product, contains three N-methylpyrrole units and binds to DNA in a 2:1 or a 1:1 stoichiometry depending on the concentration (Pelton and Wemmer, Proc. Natl. Acad. Sci. USA 86:5723, 1989).

Synthetic analogs of netropsin and distamycin have been designed to have sequence preferences that are different from their parent molecules (Dervan and Burli, Curr. Opin. Chem. Biol. 3:688, 1999). In particular, the N-methylpyrrole (Py) units of netropsin were systematically replaced with N-methylimidazole (Im) units, resulting in polyamides with altered sequence specificities from the parent compounds (Kissinger et al., Biochemistry 26:5590, 1987). Generally, G/C base pairs are recognized by the Im/Py pair, C/G base pairs are recognized by the Py/Im pair, while A/T and T/A base pairs are recognized by the Py/Py pair. The A/T and T/A degeneracy can be broken by using a third unit, namely N-methyl-3-hydroxypyrrole amino acid (Hp). Indeed, A/T base pairs are recognized by the Py/Hp pair while T/A base pairs are recognized by the Hp/Py pair. Pairs of polyamide chains form antiparallel, side-by-side dimeric complexes with DNA molecules that include an appropriate recognition sequence.

In preferred embodiments, the two polyamide chains may be linked, e.g., by a γ-butyric acid linker to form a hairpin loop that conserves the side-by-side alignment of the Im, Py, and Hp units (Mrksich et al., J. Am. Chem. Soc. 116:7983, 1994). These hairpin structures offer increased affinity and specificity.

In yet other embodiments, the polyamides may include one or more β-alanine units, preferably designed to lie adjacent to A/T or T/A base pairs. The β-alanine units relax the curvature of the polyamides and have been shown to enhance the affinity and selectivity of polyamides that recognize long nucleotide sequences (Trauger et al., Angew. Chem. Int. Ed. 37:1421, 1998 and Trauger et al., J. Am. Chem. Soc. 120:3534, 1998). Cyclic polyamides and/or pairs of polyamide hairpins linked via flexible chains, e.g., valeric acid chains are also within the scope of the present invention (Herman et al., J. Am. Chem. Soc. 121:1121, 1999).

Molecular Probes That Include an Oligonucleotide

In yet other embodiments of the present invention, an inventive binding entity may include a molecular probe that includes an oligonucleotide.

For example, in one simple embodiment an oligonucleotide is provided that is complementary to one of the two DNA strands of a target allele (see FIG. 1).

It will be appreciated that the separation of parental genetic material using molecular probes that include an oligonucleotide will be influenced by the conditions under which hybridization is carried out. For example, in order to promote strand invasion of a DNA molecule, it is likely that elevated temperatures and duplex destabilizing buffer may be necessary. Such means of promoting hybridization between DNA strands are well known (Sambrook et al., Molecular Cloning: A Laboratory Manual, Cold Spring Harbor Press, New York, 1989 and Ausubel et al., Current Protocols in Molecular Biology, Greene Publishing Associates, New York, 1996).

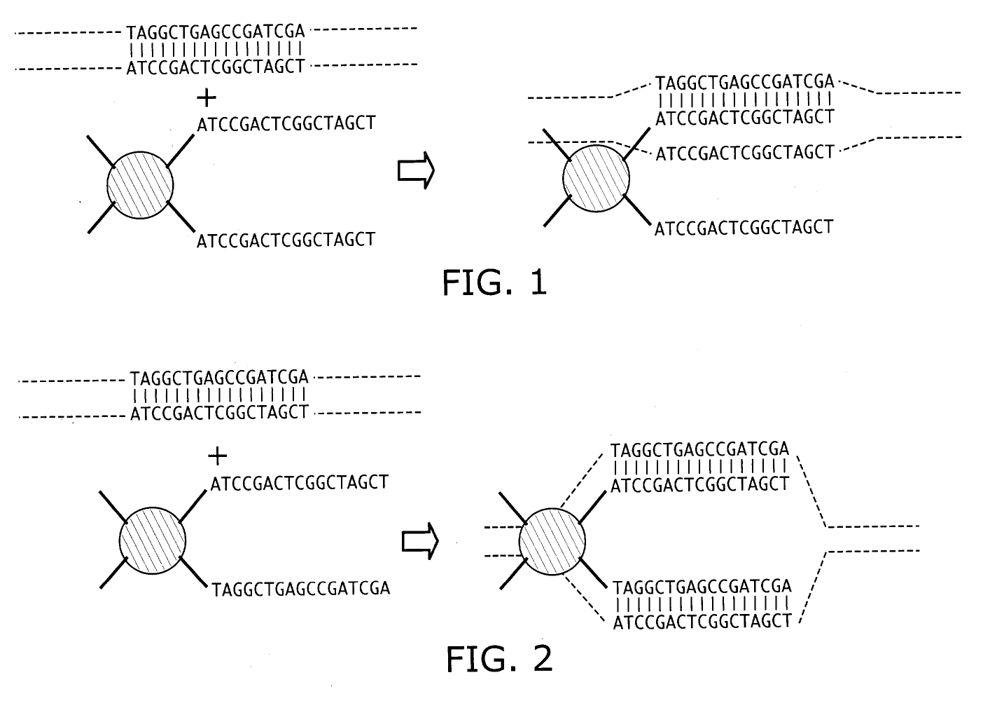

In one preferred embodiment, two complementary sequences are presented in the form of two separate oligonucleotides, each being complementary to one of the two DNA strands of a target allele (see FIG. 2).

In yet another preferred embodiment, the two complementary sequences are presented together within a single oligonucleotide separated by a short linker sequence (see FIG. 3). Without limitation, it is predicted that intramolecular base pairs may form between the complementary strands of the oligonucleotide to generate a stable hairpin loop structure (see FIG. 3). However, again without limitation, it is predicted that once hybridized to the target duplex, an equivalent number of overall base pairs in the system will be maintained by hybridization of the complementary strands of the oligonucleotide to the complementary strands of the target allele. Moreover, it is likely that the intramolecular site contiguity of the oligonucleotide will impart a cooperative behavior on its hybridization to the target duplex and hence increase the overall hybridization specificity. Without limitation, this theory is solidified by the observation that tethered oligonucleotides, which are complementary to two single stranded sequences that are in close proximity to one another, can exhibit cooperative hybridization (Richardson et al., J. Am. Chem. Soc. 113:5109, 1991). Similarly, separate oligonucleotides that hybridize to contiguous target site sequences can also show cooperative hybridization properties (Kutyavin et al., FEBS Lett. 238:35, 1988).

In order to reduce the formation of hairpin loops in an oligonucleotide that includes two complementary strands in certain preferred embodiments the oligonucleotide may include an unstructured nucleic acid (UNA; see European Patent Application No. EP1072679). In general, UNAs are oligonucleotides composed of one or more pairs of nucleotide bases (e.g., A′/T′ and G′/C′) that are unable to form stable base pairs with one another (e.g., A′≠T′ and G′≠C′) yet are able to form stable pairs with other nucleotides (e.g., A′=T; T′=A; G′=C and C′=G). UNAs have reduced levels of secondary structure compared to oligonucleotides of the same nucleotide sequence that contain only naturally occurring bases. UNAs have reduced levels of secondary structure because of their reduced ability to form intramolecular hydrogen bond base pairs between regions of complementary sequence. Preferred UNAs, however, retain the ability to form intermolecular hydrogen bond base pairs with other nucleic acid molecules.

Examples of known nucleotide bases that can be utilized in producing UNAs include 2,6-diaminopurine (D) and 2-thiothymidine (S). It is well known that adenine (A) naturally base pairs with thymidine (T) (A=T) or uridine (U) (A=U). However, A can also form a stable base pair with S (A=S) and T can also form a stable base pair with D (D=T). However, D cannot form a stable base pair with S (D≠S). Likewise, both guanosine (G) and inosine (I) can form stable base pairs with C (G=C and I=C), whereas pyrrolo-pyrimidine (X) can only form a stable base pair with G (G=X and I≠X) (Woo et al., Nucleic Acids Res. 24:2470, 1996).

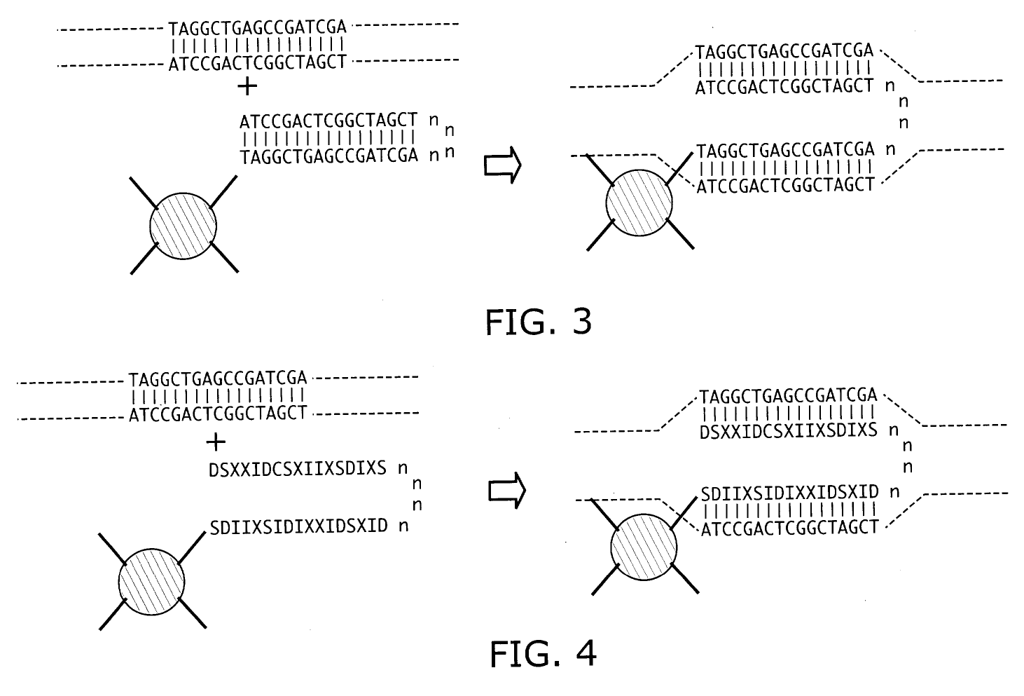

An exemplary 38 mer UNA sequence corresponding to a 10 base pair hairpin loop structure and composed of 2,6-diaminopurine, 2-thiothymidine, guanosine and cytidine, has been shown to exhibit a melting temperature about 24° C. lower than that of its DNA counterpart. Most importantly, a short 7 mer oligonucleotide which is complementary to the stem region of a hairpin loop structure is only able to hybridize to the UNA version of the sequence (European Patent Application No. EP1072679). By utilizing UNA oligonucleotides (e.g., containing D, S, I, and X) the hairpin structure inherent to the oligonucleotide sequence that includes two complementary strands should be eliminated and thus better able to strand invade a target allele (see FIG. 4).

It will also be appreciated that in the case of large molecules such as chromosomes, hybridization is preferably performed using oligonucleotides that are able to strand invade DNA molecules under conditions where the DNA is in a native or semi-denatured state. For smaller chromosome fragments, e.g., less than 5 kb in length, it may be permissible to use more denaturing conditions.

A number of approaches that have been directed toward achieving hybridization using minimal denaturing conditions. For example, it has been shown that the addition of RecA or similar proteins can promote strand invasion (Norirot et al., J. BioL Chem. 273:12274, 1998). The addition of denaturants, such as formamide, along with RNA oligonucleotides can create stable RNA-DNA hybrids known as R-loops that prime the initiation of replication in E. coli cells (Chen et al., Proc. NatL. Acad. Sci. USA 90:4206, 1993).

It is also known that certain modified oligonucleotides can promote strand invasion. These include oligonucleotides that possess uncharged backbone structures, such as peptide nucleic acids (PNAs). PNAs possess a nonionic backbone in which the deoxyribose linkages have been replaced by N-(2-aminoethyl) glycine units. The uncharged nature of the PNA inter-nucleotide linkages increases their affinity for complementary sequences under conditions of low ionic strength and increases the rate of their hybridization (Ishihara et al., J. Am. Chem. Soc. 121:2012, 1999). PNAs have association constants as much as 500 times greater than that of unmodified oligonucleotides (Iyer et al., J. Biol. Chem. 270:14712, 1995). PNAs linked to cationic proteins or peptides show annealing association rates as much as 12,000 times greater than that of unmodified oligonucleotides (Iyer et al., J. Biol. Chem. 270:14712, 1995 and Zhang et al., Nucleic Acids Res. 28:3332, 2000). The hybridization efficiency of PNAs is highest for complementary regions that have A-T rich inverted repeat sequences (Ishihara et al., J. Am. Chem. Soc. 121:2012, 1999). The annealing of PNAs to target DNA molecules show a clear temperature and salt dependence (Zhang. et al., Nucleic Acids Res. 28:3332, 2000). While these data together indicate that a target's duplex stability plays a critical role in determining the PNA annealing rate, it has been suggested that other duplex sequence motifs that can adopt non-B form conformations or assume partially single stranded structures, such as transcription promoter regions, could also be targeted for strand invasion by PNAs. Finally, it has been shown that biotin labeled PNAs can be used to affinity capture plasmid DNA using streptavidin-coated beads (Zhang et al., Nucleic Acids Res. 28:3332, 2000).

Other modified oligonucleotides known as locked nucleic acids (LNAs) have superior duplex stabilizing properties and enhanced strand invasion properties (Wengel et al., Nucleosides and Nucleotides 18:1365, 1999 and Kvaerno et al., Chem. Commun. 7:657, 1999). This property is attributed to the fact that the 2′-O, 4′-C-methylene bridge of LNAs conformationally restricts the ribose ring which induces an entropically favored duplex with one strand of the DNA target.

Multivalent Molecular Probes

In certain embodiments, a binding entity may include a molecular probe that binds preferentially to a specific combination of neighboring alleles. A suitable molecular probe could, for example, be prepared by linking two or more molecular probes as described above. For example, in certain embodiments, the linked molecular probes may bind to neighboring alleles on maternal or paternal genetic material. The arrangement of linked molecular probes may, for example, align with the arrangement of SNP sites (e.g., sequentially or conformationally) in neighboring alleles so that the linked molecular probes are able to contact and bind their respective target alleles simultaneously. Methods of linking together multiple polypeptides, small molecules, and/or oligonucleotides described herein are known in the art, see, for example, Pardridge, Pharmacol. Toxicol. 71:3, 1992; Dervan and Bürli, Curr. Opin. Chem. Biol. 3:688, 1999; and U.S. Pat. No. 5,908,626.

For example, multiple polypeptides may be included within a single recombinant polypeptide with peptide linkers of appropriate length separating each polypeptide region of interest. Preferably the peptide linkers are flexible, allowing the polypeptide regions to flex in relation to each other such that they can bind to neighboring polymorphic regions simultaneously. Typically, the peptide linkers include stretches of glycine and serine residues with some glutamic acid or lysine residues interspersed for solubility. Similarly multiple polyamide hairpin loops may be linked via flexible linkers, e.g., a valeric acid linker. Multiple oligonucleotides may be linked as a single continuous polynucleotide molecule with each oligonucleotide of interest separated by an appropriate stretch of “spacer” nucleotides (e.g., a polyadenosine stretch) so that binding to neighboring polymorphic regions occurs simultaneously.

2. Methods and Devices

In order to simplify the written description of the present invention, the remainder of the present application will discuss the inventive methods and devices as they may be used to separate homologous chromosomes. However, it is to be understood and will be appreciated from the foregoing discussion that the methods and devices are not limited to such narrow embodiments and that they may be used in a broader context and in particular to separate any homologous maternal and paternal genetic material as defined herein.

In addition, it is to be understood that in certain embodiments, the methods of the present invention may be used to separate pairs of homologous chromosomes when these are present in a sample that includes other non-homologous chromosomes (e.g., when the sample includes a full set of chromosomes from a diploid individual). It is also to be understood that, in other embodiments, the inventive methods may be used to separate pairs of homologous chromosomes after these have been extracted from a broader mixture of chromosomes. It will we be appreciated that a variety of methods exist for achieving this initial extraction step, e.g., without limitation methods that are based on differences in size and/or sequence content (e.g., CA/GT).

(a) Separation Methods When a Differentiating SNP Site is Known

In one general aspect, the present invention provides methods for separating homologous chromosomes based on prior knowledge of the nature of the SNPs that differentiate them.

Methods for Determining a Differentiating SNP Site

In order to determine whether or not an SNP site that differentiates the two homologous chromosomes exists, any SNP genotyping method may be used. For example, without limitation a preliminary SNP analysis can be performed using a TAQMANTM™ assay (available from PE Biosystems of Foster City, Calif.), an INVADERTM™ assay (available from Third Wave Technologies of Madison, Wis.), a READITTM™ assay (available from Promega of Madison, Wis.), or one of the other numerous, standard assays that require preliminary PCR. Additionally or alternatively, one may perform a preliminary SNP analysis using atomic force spectroscopy as described by Woolley et al. in Nat. Biotechnol. 18:760, 2000 and/or mass spectroscopy as described by Sauer and Gut in J. Chromatogr. B Analyt. Technol. Biomed. Life Sci. 782:73, 2002. For a general review of SNP genotyping methods, see, for example “Technologies for the Analysis of Single-Nucleotide Polymorphisms: An Overview” by Grant and Phillips in Pharmacogenomics, Volume 113, Chapter 10, Pages 183-190, Ed. by Kalow, Meyer and Tyndale, Marcel Dekker, 2001.

In general, a series of SNP analyses may be devised to provide a high statistical probability of finding at least one differentiating SNP site. The number and nature of analyses that are required may be determined by the number of SNPs that are known to occur in the homologous chromosomes of interest combined with knowledge of their statistical frequency of occurrence.

For example, if a pair of homologous chromosomes are known to include a set of 10 SNP sites, each of which exists in one of two variations (i.e., alleles) that have an equal probability of occurrence, then there is a greater than 99.9% probability, i.e., (2²⁰−2¹⁰)/2²⁰ that at least one of the SNP sites differs within the pair. It will be appreciated that a preliminary analysis of these 10 SNP sites would therefore have a 99.9% chance of providing a differentiating SNP site that could then be used as a basis for selecting a suitable molecular probe and thence separating the homologous chromosomes.

More generally for a set of n SNP sites, each of which exists in one of two alleles that again have an equal probability of occurrence, the probability of finding at least one differentiating SNP site in the pair equals (2²ⁿ−2ⁿ)/2²ⁿ, i.e., 50%, 75%, 87.5%, 93.8%, 96.9%, 98.4%, 99.2%, 99.6%, 99.8%, 99.9%, 99.999999%, for n=1, 2, 3, 4, 5, 6, 7, 8, 9, 10, 20, respectively.

Even more generally, for a set of n SNP sites, each of which exists in one of two alleles having unequalprobability of occurrence (e.g., if one allele is found in 99% of the population (p=0.99) and the other allele is found in only 1% of the population (q=0.01)), the probability of finding at least one differentiating site in the pair equals (R²ⁿ−Rⁿ)/R²ⁿ, where R=1/(1−f_{genotype}) and f_{genotype}=2pq is the average frequency with which the two alleles occur together in the population (i.e., the average heterozygous genotype frequency). For example, for a set of n SNPs all having an average heterozygous genotype frequency f_{genotype}=0.40, where R=1.6667, the resulting probability of finding at least one differentiating SNP site in the pair is equal to 40%, 64%, 78.4%, 87.0%, 92.2%, 95.3%, 97.2%, 98.3%, 99.0%, 99.4%, 99.99% for n=1, 2, 3, 4, 5, 6, 7, 8, 9, 10, 20, respectively. A person skilled in the art would readily recognize that these calculation can be extended to cover more complex embodiments, e.g., situations in which three or four alleles exist for a particular SNP site and/or thefgenotype values for the different n SNP sites are not all equal.

In general, estimating the expected heterozygous genotype frequencies (i.e., f_{genotype}=2pq values) for a defined set of SNP sites requires knowing the frequencies with which different alleles of those SNP sites occur in a given population (i.e., the p and q values). Current estimates place the number of SNPs having an allele frequency of >1% at about 11 million (Kruglyak and Nickerson Nature Genetics, 27:234, 2001). Importantly, however, as the expected allele frequency increases, the number of SNPs having that frequency decreases. For example, the number of SNPs having allele frequencies of >20%, >30%, and >40% is estimated to be about 3, 2, and 1 million.

In general, using the above allele frequency values as a benchmark, one can use the Hardy-Weinberg equation (see below) to calculate the expected heterozygous genotype frequencies for each range of allele frequencies within the population. It is worth pointing out that this type of analysis assumes that: the population is large to ensure no sampling errors; that mating between individuals is random; that there exists no additional mutations or mutational equilibrium; that there exists no selection for a given genotype; and that all genotypes reproduce with equal success (Przeworski et al., Trends Genet. 16:296, 2000). For the Hardy-Weinberg equation: p²+2pq+q²=1, where p is the frequency of the A allele, q is the frequency of the a allele, p² is the predicted frequency of the AA genotype, 2pq is the predicted frequency of the Aa genotype, and q² is the predicted frequency of the aa genotype. Table 1 below gives the predicted frequencies for the two homozygous (aa and AA) and single heterozygous (Aa) genotypes for various allele frequencies (i.e., p and q values) within the population. It is clear from this analysis that as the allele frequencies tend towards equal values in a population, this results in a higher frequency of heterozygous individuals within that population (i.e., asp and q tend towards 50%, 2pq increases).

TABLE 1

| | |
|---|---|
| Allele Frequency | Genotype Frequency |

| p (A) | q (a) | p² (AA) | q² (aa) | 2pq (Aa) |
|---|---|---|---|---|
| 1% | 99% | 0.01% | 98.01% | 1.98% |
| 2% | 98% | 0.04% | 96.04% | 3.92% |
| 5% | 95% | 0.25% | 90.25% | 9.50% |
| 10% | 90% | 1.00% | 81.00% | 18.00% |
| 20% | 80% | 4.00% | 64.00% | 32.00% |
| 30% | 70% | 9.00% | 49.00% | 42.00% |
| 40% | 60% | 16.00% | 36.00% | 48.00% |
| 50% | 50% | 25.00% | 25.00% | 50.00% |

Table 2 below was obtained by calculating values of (R²ⁿ−Rⁿ)/R²ⁿ where R =1/(1−f_{genotype}) using selected values of f_{genotype}=2pq from Table 1 above. As summarized in Table 2, a higher frequency of heterozygous genotypes (Aa) results in a decrease in the number of total SNP sites needed to ensure that at least one SNP site is differentiating between the pair of homologous chromosomes. Importantly, however, even when using SNP sites that have a lower average allele frequency within the population (e.g., p=10% in Table 1) which result in a lower average heterozygous genotype frequency (e.g., f_{genotype}=2pq=18% in Table 1), one still has a >99% probability of finding at least one differentiating site within a set of n=25 SNPs (see Table 2). Importantly, an allele frequency of 10% is close to the frequency that would be expected if the individual SNPs were chosen at random (see Kruglyak and Nickerson Nature Genetics, 27:234, 2001).

TABLE 2

| | Heterozygous Genotype Frequency (f_{genotype}) | | | | | | | |
|---|---|---|---|---|---|---|---|---|
| n | 1.98 | 3.92 | 9.50 | 18.00 | 32.00 | 42.00 | 48.00 | 50.00 |
| 1 | 1.98% | 3.92% | 9.50% | 18.00% | 32.00% | 42.00% | 48.00% | 50.00% |
| 2 | 3.92% | 7.69% | 18.10% | 32.76% | 53.76% | 66.36% | 72.96% | 75.00% |
| 3 | 5.82% | 11.31% | 25.88% | 44.86% | 68.56% | 80.49% | 85.94% | 87.50% |
| 4 | 7.69% | 14.78% | 32.92% | 54.79% | 78.62% | 88.68% | 92.69% | 93.75% |
| 5 | 9.52% | 18.12% | 39.29% | 62.93% | 85.46% | 93.44% | 96.20% | 96.88% |
| 6 | 11.31% | 21.33% | 45.06% | 69.60% | 90.11% | 96.19% | 98.02% | 98.44% |
| 7 | 13.06% | 24.42% | 50.28% | 75.07% | 93.28% | 97.79% | 98.97% | 99.22% |
| 8 | 14.78% | 27.38% | 55.00% | 79.56% | 95.43% | 98.72% | 99.47% | 99.61% |
| 9 | 16.47% | 30.23% | 59.28% | 83.24% | 96.89% | 99.26% | 99.72% | 99.80% |
| 10 | 18.13% | 32.96% | 63.15% | 86.26% | 97.89% | 99.57% | 99.86% | 99.90% |
| 11 | 19.75% | 35.59% | 66.65% | 88.73% | 98.56% | 99.75% | 99.92% | 99.95% |
| 12 | 21.34% | 38.11% | 69.82% | 90.76% | 99.02% | 99.86% | 99.96% | 99.98% |
| 13 | 22.89% | 40.54% | 72.68% | 92.42% | 99.34% | 99.92% | 99.98% | 99.99% |
| 14 | 24.42% | 42.87% | 75.28% | 93.79% | 99.55% | 99.95% | 99.99% | 99.99% |
| 15 | 25.92% | 45.11% | 77.63% | 94.90% | 99.69% | 99.97% | 99.99% | 100.00% |
| 16 | 27.38% | 47.26% | 79.75% | 95.82% | 99.79% | 99.98% | 100.00% | 100.00% |
| 17 | 28.82% | 49.33% | 81.68% | 96.57% | 99.86% | 99.99% | 100.00% | 100.00% |
| 18 | 30.23% | 51.32% | 83.42% | 97.19% | 99.90% | 99.99% | 100.00% | 100.00% |
| 19 | 31.61% | 53.22% | 84.99% | 97.70% | 99.93% | 100.00% | 100.00% | 100.00% |
| 20 | 32.97% | 55.06% | 86.42% | 98.11% | 99.96% | 100.00% | 100.00% | 100.00% |
| 21 | 34.29% | 56.82% | 87.71% | 98.45% | 99.97% | 100.00% | 100.00% | 100.00% |
| 22 | 35.59% | 58.51% | 88.88% | 98.73% | 99.98% | 100.00% | 100.00% | 100.00% |
| 23 | 36.87% | 60.14% | 89.93% | 98.96% | 99.99% | 100.00% | 100.00% | 100.00% |
| 24 | 38.12% | 61.70% | 90.89% | 99.15% | 99.99% | 100.00% | 100.00% | 100.00% |
| 25 | 39.34% | 63.20% | 91.75% | 99.30% | 99.99% | 100.00% | 100.00% | 100.00% |

Once one or more SNP sites that differentiate the homologous chromosomes have been determined as described above, the inventive methods may use this information to separate the chromosomes. For example, in a particularly simple embodiment of the present invention, if it is known that two homologous chromosomes differ at a particular SNP, then a simple separation can be performed using a binding entity that includes a molecular probe for one of the differentiating alleles.

Using Molecular Probes Alone or Molecular Probes That are Associated with “Electrophoretic” Tags

In certain embodiments, the binding entity that is used to separate a pair of homologous chromosomes may include a molecular probe alone or a molecular probe associated with an “electrophoretic” tag. According to such embodiments, once the molecular probe binds with a member of a pair of homologous chromosomes, the members of the pair are separated by electrophoresis. Indeed, although electrophoretic separation of homologous chromosomes or fragments is typically not possible because their electrophoretic mobilities are identical or near identical, the binding of a molecular probe can lead to alteration of electrophoretic mobility, and hence separation. Alteration of DNA mobility after binding to proteins such as ZFP is a known phenomenon (e.g., see Lai et al., J. Biol. Chem. 270:25266, 1995). Typically such changes in mobility (gel shifts) are observed with relatively short pieces of DNA. However, by associating the molecular probes with suitable “electrophoretic” labels, these effects should be observable on larger DNA fragments and on intact chromosomes. In general, any label that causes the electrophoretic mobility to be sufficiently altered to allow bound and unbound homologous chromosomes to become separated by electrophoresis is encompassed by the present invention. Typically, suitable “electrophoretic” labels will alter the charge, size, or electrophoretic alignment of chromosomes (e.g., see Viovy, Mol. Biotechnol. 6:31, 1996).

Using Molecular Probes That are Associated With Affinity Tags

In certain embodiments the binding entity that is used to separate a pair of homologous chromosomes may include an affinity tag that is associated with the molecular probe. According to such embodiments, when the molecular probe binds with a chromosome of interest an affinity tagged molecular probe-chromosome complex is formed. The complex can then be extracted from the mixture of homologous chromosomes using a capture agent that is complementary with the affinity tag (i.e., that binds the affinity tag).

It will be appreciated that any affinity tag known in the art may be used as long as a complementary capture agent exists. In general, suitable affinity tag/capture agent pairs include any ligand/receptor pair such as antibody/antigen, protein/co-factor, and enzyme/substrate pairs. Besides the commonly used biotin/avidin pair, these include without limitation, biotin/streptavidin, digoxigenin/anti-digoxigenin, FK506/FK506-binding protein (FKBP), rapamycin/FKBP, cyclophilin/cyclosporin, and glutathione/glutathione transferase pairs. Other suitable ligand/receptor pairs would be recognized by those skilled in the art, e.g., monoclonal antibodies paired with an epitope tag such as, without limitation, glutathione-S-transferase (GST), c-myc, FLAG®, and maltose binding protein (MBP) and further those described in Kessler pp. 105-152 of “Advances in Mutagenesis” Ed. by Kessler, Springer-Verlag, 1990; “Affinity Chromatography: Methods and Protocols (Methods in Molecular Biology)” Ed. by Pascal Baillon, Humana Press, 2000; and “Immobilized Affinity Ligand Techniques” by Hermanson et al., Academic Press, 1992. Phenylboronic acid complexes may also be used for preparing affinity tag/capture agent pairs (e.g., as described in U.S. Pat. No. 5,594,151). In addition, polyA/polyT pairs may be used.

It will be appreciated that any known method for isolating affinity tags may be used to extract an affinity tagged molecular probe-chromosome complex (e.g., those described in the references provided above). For example, without limitation, a solid phase, e.g., a slide, a membrane, a gel, beads, particles, etc. that is associated with complementary capture agents may be used. According to such an exemplary embodiment, the complexes become bound by the solid phase when the mixture is passed over or through the solid phase. The chromosomes are then released and recovered from the solid phase using standard elution techniques, i.e., by weakening the affinity between the chromosome and molecular probe.

The foregoing description has focused on the use of a single molecular probe that binds preferentially to a first member of a pair of homologous chromosomes. It is to be understood that the methods of the present invention encompass the use of additional molecular probes that bind preferentially to the first member of the pair and/or the use of molecular probes that bind preferentially to the second member.

In particular, multiple molecular probes that target different known heterozygous SNP sites of a homologous pair of chromosomes could be used in series to reduce the possibility of a separation failure. Alternatively or additionally multiple molecular probes used in series may be used to separate a single chromosome using multiple criteria.

Used simultaneously, multiple molecular probes could be used to separate multiple pairs simultaneously. It will be appreciated that when multiple different molecular probes are used simultaneously it is preferred that there be some means of differentiating them. This may be achieved in a variety of ways. For example, molecular probes could be associated with different affinity tags that allow them to be captured and separated using different capture agents.

In certain preferred embodiments, several parameters of the invention may be optimized in order to minimize shear forces on the chromosomes that are being separated by any of the above methods. This is particularly important when using the methods of the present invention to separate large chromosomes in order to avoid fragmentation.

In one preferred embodiment of the invention, under the required separation conditions, the shear forces during the separation process are minimized by embedding the chromosomes into a small gel plug, preferably a thermal-stable gel plug before contacting the chromosomes with a binding entity. Loading and manipulating large polynucleotides such a chromosomes from gel plugs is known in the art (Schwartz et al., Cell 37:67, 1984; Anand, Trends Genet. 2:278, 1986; and Smith et al., Methods in Enzymology 155:449, 1987). A solution of affinity tagged molecular probes could be diffused into a gel plug containing the chromosomes. Within the gel, the freely diffusing molecular probes would bind to the appropriate target allele, after which a separation could be performed by electrophoretically driving the molecular probe-chromosome complex through or over a solid phase that is associated with the appropriate capture agent. The use of a gel as a solid phase is a preferred embodiment (e.g., see Akerman, J. Am. Chem. Soc. 121:7292, 1999; Anada et. al., Electrophoresis 23:2267, 2002; Muscate et. al. Anal. Chem. 70:1419, 1998; and Baba, J. Biochem. Biophys. Methods 41:91, 1999).

In another preferred embodiment, instead of relying upon gravity or positive fluid pressure to drive chromosomes past or through a solid phase, shear forces may be reduced by imbedding the solid phase into a gel and driving the chromosomes through the gel using some form of electrokinetic force, similar to that employed in pulse-field electrophoresis methods, e.g., field alteration gel electrophoresis (FAGE) (Schwartz et al., Cell 37:67, 1984; Carle et al., Science 232:65, 1986; and Viovy, Review of Modern Physics, 72:813, 2000).

Using Molecular Probes That Are Associated With a Solid Phase

In certain embodiments, the binding entity that is used to separate a pair of homologous chromosomes may include one or more molecular probes that are associated with a solid phase. Any form of association, whether direct or indirect, may be employed in the practice of the present invention so long as it is sufficient to associate the molecular probe with the solid phase as described herein. As is well known in the art, a variety of methods are known for associating the molecular probes of the present invention with the surfaces of a variety of solid phases, including but not limited to glass surfaces, ceramic surfaces, metal surfaces, plastic surfaces (e.g., see “Affinity Chromatography: Methods and Protocols (Methods in Molecular Biology)” Ed. by Pascal Baillon, Humana Press, 2000 and “Immobilized Affinity Ligand Techniques” by Hermanson et al., Academic Press, 1992). Without limitation, one or more molecular probes may be associated with one or more slides, membranes, beads, particles, etc. In certain embodiments, one or more molecular probes may be associated with a gel. As defined herein, a “gel” encompasses agarose gels and cross-linked polyacrylamide gels but also solutions of polymers that can act like a gel for electrophoretic purposes. Methods for associating molecular probes with gels and suitable polymers are known in the art, e.g., see Akerman, J. Am. Chem. Soc. 121:7292, 1999; Anada et. al., Electrophoresis 23:2267, 2002; Muscate et. al. Anal. Chem. 70:1419, 1998; and Baba, J. Biochem. Biophys. Methods 41:91, 1999.

According to such embodiments, when a chromosome of interest binds the molecular probe it becomes associated with the solid phase. If the solid phase is a slide, a membrane, or a gel, the chromosome of interest can be separated from the remainder of the mixture by passing the mixture over or through the solid phase. The same applies if the solid phase includes a collection of particles or beads that have been packed into an affinity column or a microfluidic matrix. As is well known in the art, the chromosomes can be recovered by eluting them from the solid phase using an eluting solution that weakens the affinity between the molecular probe and the chromosome of interest.

In certain embodiments, an inventive binding entity may include a suspension of beads or particles that are associated with molecular probes. In such embodiments, the suspension of beads or particles is contacted with the pair of homologous chromosomes. The molecular probes bind the chromosome with the appropriate allele thereby forming a bead/particle-molecular probe-chromosome complex. The beads or particles are then extracted from the mixture. The extraction step might involve filtering, decanting or centrifuging the beads or particles; isolating them using a magnetic field (e.g., if the beads are paramagnetic); isolating them using a complementary capture agent (e.g., if the beads or particles are also associated with an affinity tag); or separating them using flow cytometry.

A variety of beads and particles (in particular those made of polystyrene and silica) are available from Bangs Laboratories of Fishers, Ind. or Duke Scientific Corp. of Palo Alto, Calif. In addition, paramagnetic beads are available under the trademarked name BIOMAG™ from Polysciences of Warrington, Pa. and under the trademarked name DYNABEAD™ from Dynal Biotech of Oslo, Norway. A variety of beads that are associated with affinity tags are also available commercially, e.g., from Spherotech of Libertyville, Ill.; Polysciences of Warrington, Pa.; Qiagen of Valencia, Calif.; Quantum Magnetics of Madison, Conn.; Dynal Biotech of Oslo, Norway; Biosource International of Camarillo, Calif.; Calbiochem of San Diego, Calif.; and Rockland Immunochemicals of Gilbertsville, Pa.

Again, the foregoing description has focused on the use of a single molecular probe that binds preferentially to a first member of a pair of homologous chromosomes. It is to be understood that, in this context, the methods of the present invention also encompass the use of additional molecular probes that bind preferentially to the first member of the pair and/or the use of molecular probes that bind preferentially to the second member.

In particular, multiple molecular probes that target different known heterozygous SNP sites of a homologous pair of chromosomes could be used in series to reduce the possibility of a separation failure. Alternatively or additionally multiple molecular probes used in series may be used to separate a single chromosome using multiple criteria.

Used simultaneously, multiple molecular probes could be used to separate multiple pairs simultaneously. It will be appreciated that when multiple different molecular probes are used simultaneously it is preferred that there be some means of differentiating them. This may be achieved in a variety of ways. For example, molecular probes that are associated with beads could be associated with different types of beads (e.g., beads that are paramagnetic and non-paramagnetic, different sized beads, beads with different densities, beads that are labeled with different affinity tags, etc.).

Using Molecular Probes That Are Associated With Detectable Labels

In certain embodiments, the binding entity that is used to separate a pair of homologous chromosomes may include one or more molecular probes that are associated with detectable labels. In certain embodiments, the detectable label is directly detectable. In other embodiments, the detectable label is indirectly detectable, e.g., through combined action with one or more additional members of a signal producing system. Examples of detectable labels include radioactive, paramagnetic, fluorescent, light scattering, chemiluminescent, absorptive, and calorimetric labels.

Paramagnetic labels of interest include labels with paramagnetic ions, e.g., chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

Fluorescent labels of interest include phycoerythin, coumarin and its derivatives, e.g., 7-amino-4-methylcoumarin, aminocoumarin, bodipy dyes, such as BODIPY™ FL, cascade blue, fluorescein and its derivatives, e.g., fluorescein isothiocyanate, Oregon green, rhodamine dyes, e.g., Texas red, tetramethylrhodamine, eosins and erythrosins, cyanine dyes, e.g., Cy3 and Cy5, macrocyclic chelates of lanthanide ions, e.g., QUANTUM DYE™, fluorescent energy transfer dyes, such as thiazole orange-ethidium heterodimer, TOTAB, dendrimeric dyes, e.g., from Genisphere of Bala Cynwyd, Pa., etc.

Also of interest are nanometer sized particle labels detectable by fluorescence commonly called “quantum dots”, e.g., those described in Chan et al., Curr. Opin. Biotechnol. 13:40, 2002. Members of the family of fluorescent proteins such as green fluorescent protein, etc. as described in Matz et al., Bioessays 24:953, 2002 are of particular interest for use with molecular probes that include polypeptides, e.g., in the form of fusion polypeptides as is known in the art. Plasmon resonance particles, e.g., from Genicon Sciences of San Diego, Calif. can also be used for fluorescent detection. Other nanoparticles that are detectable by light scattering include nanogold particles, e.g., from Nanoprobe of Yaphank, N.Y.

Chemiluminescent labels of interest include enzymes that are capable of converting a substrate to a chromogenic product, e.g., alkaline phosphatase, horseradish peroxidase, and the like.

In other embodiments, labeled nucleotides can be incorporated within an molecular probe by PCR of an oligonucleotide primer that is present within the molecular probe. The use of a circular DNA template during this extension reaction (a rolling circle amplification) has been shown to be useful means of detection (e.g., see Schweitzer et al., Nature Biotechnology 20:359, 2002 and Zhong et al., Proc. Natl. Acad. Sci. USA 98: 3940, 2001). Rolling circle amplification incorporates large numbers of labels into the molecular probe, providing for signal enhancement. Such polymerase extension labeling techniques can be done prior to, or more advantageously, after the molecular probe has bound to a chromosome of interest. Multiple distinguishable labels can be incorporated via rolling circle amplification if unique primer sequences are present in different molecular probes, and the circular templates are designed to incorporate uniquely labeled dNTPs (e.g., see U.S. Pat. No. 6,054,274). For example, one circular template sequence could include just A, T, and G bases, while another circular template sequence includes just A, T, and C bases. Simultaneous incorporation during the extension reaction of e.g., Cy5 labeled dCPT and Cy3 labeled dGTP would result in two uniquely labeled molecular probes.

Additional labels of interest include those that provide for signal only when they are associated with a target sequence, where such labels include: “molecular beacons” as described in Tyagi and Kramer, Nature Biotechnology 14:303, 1996 and European Patent Application No. EP0070685. Other labels of interest include those described in U.S. Pat. No. 5,563,037; PCT Publication Nos. WO 97/17471 and WO 97/17076.

It will be appreciated that once a labeled molecular probe has bound a target chromosome within a pair of homologous chromosomes, the labeled chromosome may be visualized or detected in a variety of ways, with the particular manner of detection being chosen based on the particular label of the molecular probe, where representative detection means include, e.g., scintillation counting, autoradiography, measurement of paramagnetism, fluorescence measurement, colorimetric measurement, light emission measurement, measurement of light scattering and the like. In certain preferred embodiments, labeled chromosomes are separated based on the step of detecting.

In certain embodiments the separation step is performed by flow cytometry. In general, flow cytometry is an instrumental method that is used for the quantitative analysis and enrichment of populations of “particles” from mixed samples of interest. Flow cytometers perform quantitative analysis of almost any kind of biologically relevant “particle” including multicellular organisms, cells, nuclei and chromosomes. Flow cytometers typically measure the features of each member in a sample of interest by carrying individual members in a flow of liquid past a detector. Typically the flow cytometers are designed so that the members of a sample of interest pass through a detection zone individually.

Flow cytometry is currently widely used to identify and separate human chromosomes. Individual chromosomes can be resolved based on size and/or sequence content (e.g., CA/GT) using DNA-specific dyes. Since the size and overall sequence content of homologous chromosomes tend to be very similar (except for the X and Y chromosomes) they cannot be separated using these methods. Standard protocols typically involve preparing metaphase chromosomes from cells in culture (e.g., lymphocytes isolated from blood biopsy, stimulated with mitogen then cultured in presence of mitotic inhibitor) or other actively replicating cells. Intact metaphase chromosomes isolated from these materials can be fixed, stained and sorted for subsequent analyses.

The present invention encompasses a variety of methods for labeling, detecting, and then separating a pair of homologous chromosomes using flow cytometry. In a simplest of embodiments, a single molecular probe is used that includes a particular label. The molecular probe is contacted with the homologous chromosomes so that it can bind with the target chromosome. The mixture is then passed through a flow cytometer and the labeled chromosomes are separated from the mixture based on detection of the label. It is to be understood that the present invention may be used in conjunction with any type of flow cytometer that is suitable for sorting chromosomes or fragments thereof. Without limitation this includes those described in “Flow cytometry: First Principles” by Givan, Wiley-Liss, 2001 and “Practicalflow cytometry”by Shapiro, John Wiley & Sons, 2002. The initial mixture of chromosomes, can include without limitation, the pair of homologous chromosomes, or a mixture of the pair of homologous chromosomes and some or all other chromosomes. In some embodiments, pairs of homologous chromosomes may be separated from the mixture, for example by flow cytometry, before binding with the molecular probe.

Additionally, two different molecular probes that are associated with different labels (e.g., without limitation two labels of different color) may be used that bind preferentially to alleles on the different members of the pair. The latter embodiment will be particularly useful when both members of a homologous pair of chromosomes are to be simultaneously separated from a broader mixture.

It will further be appreciated that in certain embodiments multiple pairs of homologous chromosomes that are present in the same sample may be separated simultaneously, e.g., by using various combinations of molecular probes and detectable labels.

(b) Separation Methods When Differentiating SNP Sites Are Unknown

In another general aspect, the present invention provides methods for separating homologous chromosomes without any prior knowledge of the nature of the SNPs that differentiate them.

Instead, the statistical frequencies with which different SNPs occur in the population are used to ensure that there is a high probability that the members of the pair can be separated. In essence, the logic behind this aspect of the invention parallels the logic that was described earlier with respect to calculating the minimum number of SNP sites that need to be genotyped in order to ensure that there is a high probability that at least one differentiating SNP site can be identified (e.g., with reference to Tables 1 and 2). In the context of the present aspect, the same calculations yield the minimum number of SNP sites that need to be probed in order to ensure that there is a high probability that the members of the pair can be separated.

For example, without limitation, if five SNP sites are known to occur within the homologous chromosomes of interest, and if it is further known that these exist as one of two alleles that occur with equal probabilities (i.e., p=q=50% in Table 1, f_{genotype}=2pq=50%), then there is about 97% probability that probing for the two alleles of those five SNP sites will be sufficient to separate the homologous chromosomes (see far right column of Table 2). Removing one of the five SNP sites reduces the probability to about 94% while adding a sixth SNP site increases it to about 98.5% (see far right column of Table 2).

Using Pairs of Affinity Columns or Equivalents Thereof

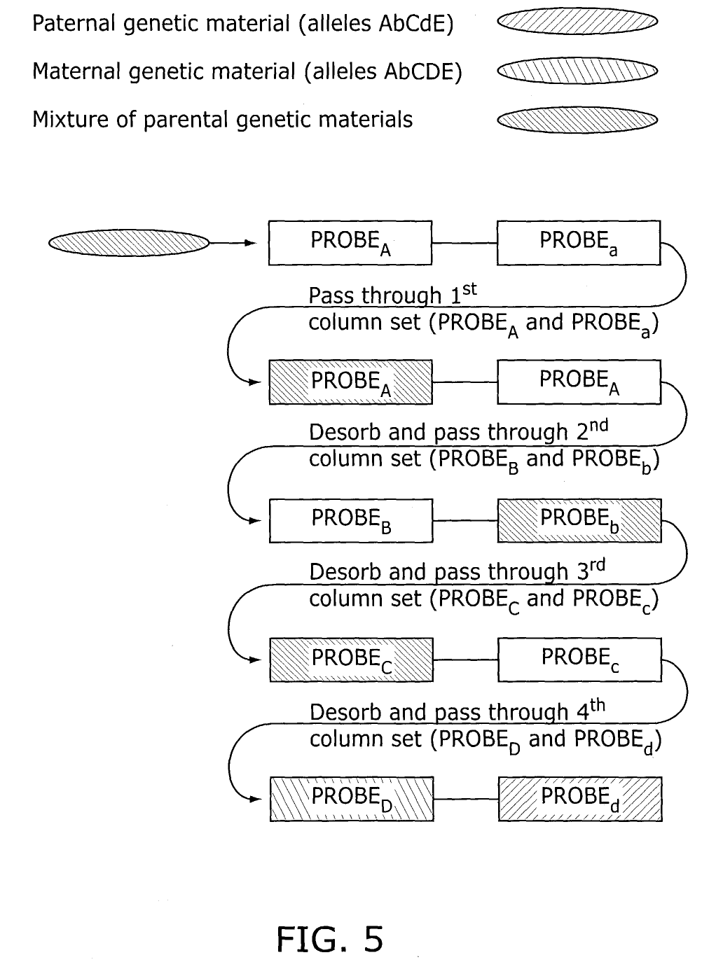

FIG. 5 illustrates one embodiment for achieving separation of homologous chromosomes based on the hypothetical described above. As illustrated, a mixture of homologous chromosomes is provided. The maternal chromosome includes alleles AbCdE. The paternal chromosome includes alleles AbCDE. Accordingly, the homologous chromosomes are homozygous AA, bb, CC, and EE but heterozygous dD.

As illustrated, the separation is accomplished by passing the pair of homologous chromosomes through a series of affinity column pairs. It is to be understood that the use of affinity column pairs is exemplary and that any equivalent structure may be used, e.g., pairs of microfluidic matrices, etc. Each affinity column pair includes a first affinity column that is designed to select for an upper case allele (e.g., A) and a second affinity column that is designed to select for a lower case allele (e.g., a). Without limitation, the affinity column may, for example, include a solid phase that is associated with molecular probes that bind preferentially to an upper case allele over the lower case allele (and vice versa).

Visualization of the position of the homologous chromosomes as they pass through each column pair may be done with a stain or intercalating dye that does not affect binding with the molecular probes. Sequential binding and elution are continued until one of the members of the pair of homologous chromosomes is retained by the first column in the pair while the other member of the pair is retained by the second column in the pair.

As illustrated in FIG. 5, for the hypothetical, the mixture of homologous chromosomes is passed through the first pair of coupled affinity columns. Since the maternal and paternal chromosomes are homozygous AA, both the paternal and the maternal chromosomes are bound by the molecular probe of column A. If the homologous chromosomes are present in a sample that includes other non-homologous chromosomes, these non-homologous chromosomes should pass through the column without becoming bound and may be diverted to waste or for further analysis. The maternal and paternal chromosomes are then eluted from affinity column A and passed through affinity columns b and B. Again, since both the parental chromosomes are homozygous bb, both the maternal and paternal chromosomes are bound by the molecular probe of affinity column b. The same outcome occurs for affinity columns c and C, where both the paternal and the maternal chromosomes bind to affinity column C, since the parental chromosomes are homozygous CC. However, on columns d and D, the maternal and paternal chromosomes are bound by different affinity columns in the coupled pair because they are heterozygous dD. The maternal and paternal chromosomes are therefore separated by their preferential affinities to the different allele-specific molecular probes. The maternal and paternal chromosomes can then be eluted from their respective affinity columns and prepared for further analysis (e.g., SNP genotyping).

It will be appreciated that the simple hypothetical that is illustrated in FIG. 5 may be extended to include more or fewer than five pairs of coupled affinity columns, and that the probability of separating two homologous chromosomes will increase as the number of pairs increases. Furthermore, it is to be understood that for each locus, one may use more than two coupled affinity columns (e.g., if four different alleles of a particular SNP site occur in a population, one could use a quartet of coupled affinity columns).

According to these embodiments, each affinity separation step can be performed manually or in an automated system that employs a series of defined columns. For example, if a series of affinity separation steps is employed, they may be performed using an integrated microfluidic chip-based system (a “microfluidic matrix”). The series of affinity columns may be linked together with the necessary bypass channels, elution buffer reservoirs, and sample collection reservoirs so that the entire process is automated. In order to reduce unwanted fragmentation of the homologous chromosomes due to shearing forces, the sample may be loaded as a gel plug and the chromosomes driven through the affinity columns using an electrokinetic force. In preferred embodiments, the microfluidic matrix may include a gel (e.g., see Akerman, J. Am. Chem. Soc. 121:7292, 1999; Anada et. al., Electrophoresis 23:2267, 2002; Muscate et. al. Anal. Chem. 70:1419, 1998; and Baba, J. Biochem. Biophys. Methods 41:91, 1999). Using this approach, the binding and elution of the chromosomes to the series of molecular probes on, e.g., a chip is determined by a combination of parameters, including the particular electrodes that are activated, local buffer conditions, and temperature.

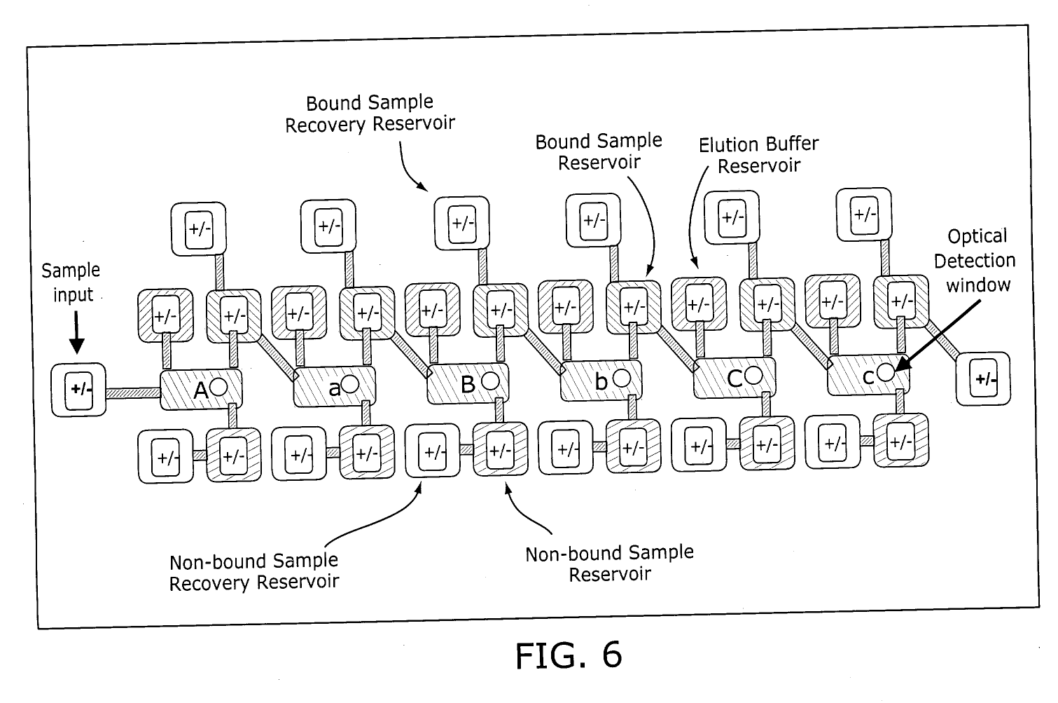

For example, FIG. 6 illustrates an automated inventive system where a sample input vessel is connected to a series of columns, each containing molecular probes to different alleles of a pair of homologous chromosomes. Each column is connected to a bound sample recovery reservoir for storing samples that were bound and subsequently recovered (i.e., eluted) from a particular column prior to analysis; a bound sample reservoir for storing a sample that bound to a particular column prior to applying the sample to a sequential column in the series; an elution buffer reservoir for storing the elution buffer; an unbound sample recovery reservoir for storing samples that did not bind to a particular column prior to analysis; and an unbound sample reservoir for storing a sample that did not bind to a particular column prior to applying the sample to a sequential column in the series. Each column may further include an optical detection window to monitor passage of chromosomes through the column.

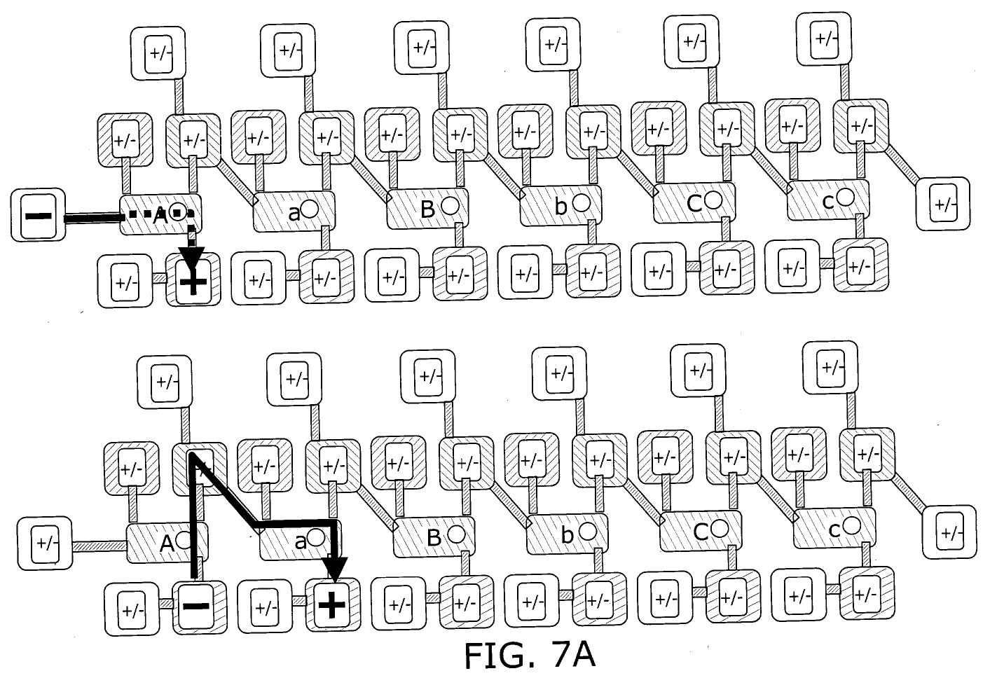

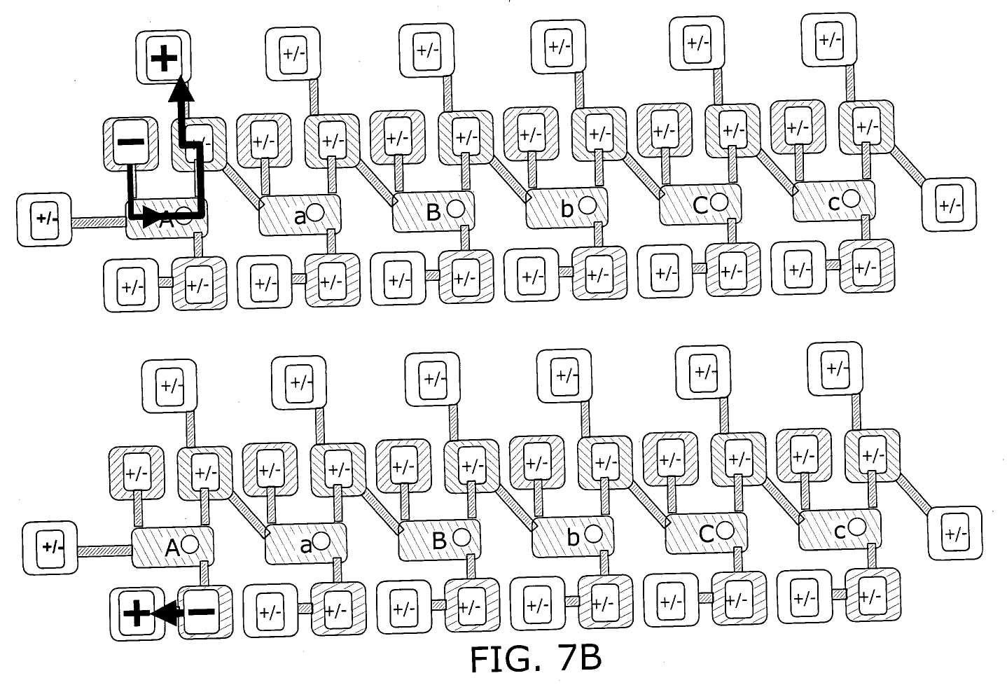

FIGS. 7A and 7B, illustrate an operational embodiment of the system that is depicted in FIG. 6. In a first step the sample is loaded into the sample input site and a net current (in the direction of the arrow) is applied between the indicated electrodes (see FIG. 7A, upper). The homologous chromosomes will either both bind to matrix A (indicates homozygous AA), both flow through to the unbound sample reservoir (indicates homozygous aa), or one member will become bound to matrix A while the other flows through to the unbound sample reservoir (indicates heterozygous Aa).

If both of the chromosomes bind to matrix A (indicates homozygous AA), then they are subsequently eluted into the bound sample recovery reservoir for further analysis (see FIG. 7B, upper). From here they can be driven through matrix a (where neither chromosome should bind) and then on to matrices B, b, C, c, etc. until they are separated (not shown).

If none of the chromosomes bind to matrix A (indicates homozygous aa) they will be driven to the unbound sample reservoir (see FIG. 7A, upper). From here, the chromosomes can then be driven through matrix a where they should all bind (see FIG. 7A, lower) and then on to matrices B, b, C, c, etc. until they are separated (not shown).

If one half of the sample (indicates heterozygous Aa) does not bind to site A and is driven to the unbound sample reservoir, then this half (i.e., with allele a) can be recovered by driving it to the unbound sample recovery reservoir (see FIG. 7B, lower).

In other embodiments, instead of using coupled affinity columns or a microfluidic matrix, a mixture of beads or particles that are associated with different molecular probes may be used. Molecular probes for different alleles of a given SNP site are associated with different types of beads or particles so that they can be distinguished. In such embodiments, the beads or particles (e.g., those for alleles A and a) are contacted with the pair of homologous chromosomes and subsequently separated based on a physical property such as magnetism, density, and/or size. Alternatively as discussed earlier, the beads or particles may be associated with an affinity tag so that they can be isolated with a complementary capture agent; or the beads or particles may be separated by flow cytometry. The process is repeated with beads or particles for alleles B and b, C and c, etc. until the members of the homologous pair of chromosomes become associated with a different bead or particle type and are thus separated from each other.

As but another example, homologous chromosomes and molecular probes may also be contacted in solution phase (i.e., without associating the molecular probes with a solid phase). Preferably the different molecular probes are labeled with different affinity tags or detectable labels that allow them to be separated as described previously.

Using a Mixed Bed Affinity Column or Equivalents Thereof

The use of affinity columns and microfluidic matrices as described above, as well as the use of molecular probes that are associated with a solid phase in general, require that the binding of the chromosome to the solid phase via the molecular probe be highly selective and relatively strong. A high selectivity ensures that the appropriate molecular probe will bind to a chromosome, while the strong binding ensures that the molecular probe, once bound to the chromosome, does not prematurely dissociate. It should be recognized that the strength of the desired binding is a function of a number of factors, including the time scale of the experiment as well as the length of the column or matrix. A shorter time scale will allow less time for equilibration, hence less dissociation. A longer column or matrix will allow for some dissociation, and hence migration of the chromosome through, but not out of, the column or matrix. In a related embodiment, a true chromatographic separation of the homologous chromosomes is performed by using binding conditions that are both highly selective and reversible, i.e., in rapid equilibrium with the surrounding liquid. In such embodiments, the binding entity may include a solid phase that is associated with a mixture of molecular probes for multiple polymorphic regions (but only one allele for each region). The solid phase may, for example, be arranged as an affinity column or microfluidic matrix. As the mixture of homologous chromosomes pass over or through the column or matrix, some chromosomes within the mixture are preferentially retarded, resulting in a relative retardation of the rate at which the chromosomes move through the column or matrix. For example, a molecular probe that binds preferentially to an allele that is found in only one of the chromosomes of the pair will preferentially retard only the one chromosome that includes that allele. Conversely, a molecular probe that binds preferentially to an allele region that is found in both members of a pair of homologous chromosomes will retard both chromosomes equally on the column or matrix.

The present embodiment encompasses the realization that proper separation of homologous chromosomes using these methods will depend on the net number of probed for heterozygous sites that differ between the homologous chromosomes; the greater the net number of differentiating heterozygous sites that are probed for, the greater the degree and likelihood of separation using a mixture of molecular probes as described above. This point is illustrated in Table 3, below, which demonstrates that pairs of homologous chromosomes with the greatest number of probed for heterozygous sites will be most clearly separated using these inventive methods and devices.

| Allelic identity of | | |
|---|---|---|
| homologous | Column including molecular probes for | |
| chromosomes | A, B, C, D, E, F, and G alleles | Outcome |
| ABCDEFG | Chromosome ABCDEFG will be retarded | Chromosomes will be easily |
| | by 7/7 of the molecular probes. | separated on the column. |
| abcdEFG | Chromosome abcdEFG will be retarded by | |
| | 3/7 of the molecular probes. | |
| ABCDEFg | Chromosome ABCDEFg will be retarded | Chromosomes will be separated |
| | by 6/7 of the molecular probes. | on the column, but with less |
| | | resolution. |
| abcDEFG | Chromosome abcDEFG will be retarded by | |
| | 4/7 of the molecular probes. | |
| ABCDEfg | Chromosome ABCDEfg will be retarded by | Chromosomes will likely not be |
| | 5/7 of the molecular probes. | separated at all on the column. |
| abCDEFG | Chromosome abCDEFG will be retarded by | Some separation may occur due |
| | 5/7 of the molecular probes. | to differences in relative |
| | | binding affinity at different |
| | | sites. |

It is also worth noting that this approach is not limited to using a homogeneously mixed bed column or matrix. For example, instead of using a mixed bed column or matrix, similar results could be obtained using a series of columns or matrices, or individual regions within a single column or matrix, each containing one or more molecular probe types.

3. Kits and Systems

The present invention further provides kits for separating the members of a pair of homologous chromosomes. The kits include at least a binding entity that can be used to separate the pair. The binding entity may include one or more molecular probes provided in dry form, in solution, associated with a solid phase, e.g., bead, particle, slide, membrane, gel, etc. In certain embodiments, the kits include a gel, or a plurality of beads or particles arranged in an affinity column or microfluidic matrix. In certain embodiments, the kit includes a plurality of pairs of columns or matrices that each select for a different version of a different polymorphic region of the pair.

In one embodiment, an inventive kit may include a first molecular probe that binds preferentially with a first allele of a first polymorphic region that is present within the homologous maternal and paternal genetic material and a second molecular probe that binds preferentially with a second allele of the same first polymorphic region.

The first and/or second molecular probes may be associated with an electrophoretic tag that alters the electrophoretic mobility of maternal or paternal genetic material that is bound by the molecular probes. Alternatively, the first and second molecular probes may be associated with first and second affinity tags, respectively. Such an inventive kit may preferably also include first and second solid phases that are associated with a capture agent for these first and second affinity tags, respectively. In other embodiments, the first and second molecular probes may be associated with different solid phases or different detectable tags.

In one embodiment an inventive kit may include third and fourth molecular probes that binds preferentially with first allele and second alleles, respectively, of a second polymorphic region that is present within the homologous maternal and paternal genetic material. According to such embodiments, the first, second, third, and fourth molecular probes may be associated with first, second, third, and fourth solid phases that are arranged within first, second, third, and fourth columns or matrices, respectively.

In other embodiments, an inventive kit might include a collection of different families of molecular probes, wherein each molecular probe within a given family binds reversibly with a specific allele of a polymorphic region that is present within the homologous maternal and paternal genetic material, and no more than one family includes a molecular probe that binds preferentially to an allele of a given polymorphic region. These molecular probes are preferably associated with one or more solid phases arranged within one or more columns or matrices.

The kits may further include one or more reagents for use in the assay to be performed with the binding entities, where such reagents include: reagents used in preparing the sample of interest, e.g., buffers, primers, enzymes, labels and the like; reagents used in the binding step, e.g., hybridization buffers; reagents used in the elution step, e.g., elution buffers; and the like.

Finally, systems that incorporate the subject kits are provided, where the systems find use in high throughput binding assays in which homologous chromosomes are separated. By the term “system” is meant the working combination of the enumerated components thereof, which components include those components listed below. Systems of the subject invention will generally include one or more binding entities, a fluid handling device capable of contacting the sample of interest and all reagents with the binding entities and delivering/removing elution fluid from the binding entities; and optionally a reader which is capable of providing identification of the location of positive binding events; and preferably a computer means which is capable of controlling the actions of the various elements of the system, i.e., when the reader is activated, when fluid is introduced and the like.

### EXAMPLES

### Example 1

### Determination of a Heterozyous SNP Site

Using the sample genomic DNA of interest, the genotype is determined at a number of SNP sites selected for their known high degree of heterozygosity. The SNP sites can be targeted to a particular chromosomal pair, if desired. The genotyping is done using any method, but homogeneous methods such as the TAQMAN™ assay (available from PE Biosystems of Foster City, Calif.) and the READIT™ assay (available from Promega of Madison, Wis.) are particularly convenient.

### Example 2

### Isolation of Specific Maternal or Paternal Chromosomes From Sample Using Flow Cytometry

Selection or Generation of Molecular Probes

Using the information obtained in Example 1, a finger protein (ZFP) is selected or prepared that binds specifically to a desired allele (G.B. Patent No. 2,360,285; U.S. Pat. No. 6,492,117; and PCT Publication No. WO 02/099084). The ZFP is fused with a FLAG® epitope tag for detection purposes (U.S. Pat. No. 4,782,137; U.S. Pat. No. 4,851,341; and U.S. Pat. No. 4,703,004).

Preparation of Chromosomes

Sample DNA is prepared as a suspension of metaphase chromosomes (Carrano et al., Proc. Natl. Acad. Sci USA 76:1382, 1979; Langlois et al., Proc. NatL. Acad. Sci USA 79:7876, 1982; and Speicher et al., Nature Genetics 12:368, 1996).

Allele-Specific Binding of ZFP to Chromosome

The epitope-tagged ZFP is incubated with the chromosomal DNA under buffer conditions that promote allele-specific binding, e.g., as described by Kim and Pabo, Proc. Natl. Acad. Sci USA 95:2812, 1998).

Epitope Tag Labeling

The suspension of chromosomes now also contains the maternal or paternal chromosome of interest specifically bound to the epitope-tagged ZFP. Biotinylated ANTI-FLAG® M2 antibody is added and allowed to bind to the FLAG® tagged ZFP, and excess unbound antibody is removed. Anti-biotin antibody coated resonant light scattering (RLS) particles (available from Genicon Sciences of San Diego, Calif.; see also Yguerabide and Yguerabide, J. Cell. Biochem. 37S:71, 2001) are added and allowed to complex the now biotin-tagged ZFP of interest. Uncomplexed RLS particles are removed.

Flow Sorting

The suspension of chromosomes, some of which now are labeled with RLS particles, and hence detectable by fluorescence, are sorted using a flow cytometer (Langlois et al., Proc. Natl. Acad. Sci USA 79:7876, 1982 and Telenius et al., Genes Chromosomes Cancer 4:257, 1992). For discussion of flow cytometry sensitivity for nanoparticles, see Ferris and Rowlen, Review of Scientific Instruments 73:2404, 2002. The labeled chromosomes of interest, which consist exclusively of one member of a homologous pair of chromosomes, are collected separately for further analysis. The remaining unlabeled chromosomes are also collected if desired.

### Example 3

### Isolation of Multiple Maternal or Paternal Chromosomes From a Sample Using Flow Cytometry

Selection or Generation of Molecular Probes

Using the information obtained in Example 1, two zinc finger proteins (ZFPs) are generated that each bind specifically to an allele on one member of a pair of homologous chromosomes (G.B. Patent No. 2,360,285; U.S. Pat. No. 6,492,117; and PCT Publication No. WO 02/099084). The two ZFPs are each expressed with different fused epitope tags attached, one with FLAG® and the other with c-myc tag (EQKLISEEDL).

Preparation of Detection Antibodies

ANTI-FLAG® and anti-c-myc antibodies are conjugated to unique primers for rolling circle amplification (Schweitzer et al. Proc. Natl. Acad. Sci USA 97:10113, 2000).

Preparation of Chromosomes

Sample DNA is prepared as a suspension of metaphase chromosomes (Carrano et al., Proc. Natl. Acad. Sci USA 76:1382, 1979; Langlois et al., Proc. Natl. Acad. Sci USA 79:7876, 1982; and Speicher et al., Nature Genetics 12:368, 1996).

Allele-Specific Binding of Epitope-Tagged ZFPs to Chromosomes

The ZFPs are incubated with the chromosomal DNA and allowed to bind allele-specifically to the chromosomes that contains the allele of interest (Kim and Pabo, Proc. Natl. Acad. Sci USA 95:2812, 1998). The suspension of chromosomes now contains a mixture of unlabeled chromosomes, chromosomes bound to a FLAG® tagged ZFP, and chromosomes bound to a c-myc tagged ZFP.

Epitope Tag Labeling

The previously prepared detection antibodies are allowed to complex with the respectively tagged chromosomes. Unbound antibodies are removed. Rolling circle amplification is performed with two different circular templates, and each amplicon is hybridized to distinct “decorator probes” that fluoresce at different wavelengths (Schweitzer et al., Proc. Natl. Acad. Sci USA 97:10113, 2000 and Gusev et al., American Journal of Pathology 159:63, 2001).

Flow Sorting

The suspension of chromosomes, some of which now are differentially labeled with a fluorescent label, and hence detectable and distinguishable by fluorescence, are sorted using a flow cytometer (Langlois, et al., Proc. Natl. Acad. Sci USA 79:7876, 1982). The labeled chromosomes of interest, which consist each of one member of a homologous pair of two different chromosomes are collected separately for further analysis. The remaining unlabeled chromosomes are also collected if desired.

### Example 4

### Isolation of Specific Maternal or Paternal 1Genomic Fragments Using Affinity Capture Electrophoresis

Selection or Generation of Molecular Probes

Using the information obtained in Example 1, two UNA-hairpins are prepared, each designed to specifically hybridize to one of the two differentiating alleles. The sequence of each UNA-hairpin is designed to maximize the difference in thermodynamic stability between the perfect double-duplexes and the corresponding mismatch double-duplexes formed by the two UNA-hairpins and the target duplex. For example, the hairpin is designed such that the allele-specific nucleotide is positioned within the middle of double duplexes formed by the UNA-hairpin and the target duplex. The relative thermodynamic stabilities for the duplexes can be estimated using standard nearest neighbor calculation methods (SantaLucia et al., Biochemistry 35:3555, 1996). For subsequent capture purposes, one of the allele specific UNA-hairpins is associated with a biotin tag while the other is associated with a digoxygenin tag.

Fragmentation of Genomic DNA

Genomic DNA is prepared in an agarose plug and digested with the rare cutting restriction endonuclease Notl. The fragments generated from human genomic DNA typically average around 1 Mb in length and can range in length from less than 100 kb to greater than several megabases (Doggett et al., Nucl. Acids. Res. 20:859, 1992)

Formation of The DNA-Probe Complex

The biotinylated and digoxygenin-containing UNAs are diffused into the gel plug containing the digested genomic DNA. Each UNA binds with the corresponding allele within the maternal or paternal homologous DNA fragment of interest. Excess UNA is diffusively removed from the gel plug.

Affinity Capture Electrophoresis

A capture gel is prepared containing two separate trap regions of immobilized strepavidin and anti-digoxygenin (Ito et al., Genet. Anal. Tech. AppL. 9:96, 1992). The agarose plug containing the DNA fragments complexed with the UNAs is loaded on to the capture gel, and an electric field is applied. The DNA migrates through the gel. The DNA fragment containing the biotinylated UNA/DNA fragment complex is retained in the strepavidin gel region while the digoxygenin-containing UNA/DNA fragment complex is retained in the anti-digoxygenin gel region. All the other genomic material is not retained by either of the two regions and can be recovered. The retained DNA is recovered from the gel and used for further analysis.

### Example 5

### Isolation of Specific Maternal or Paternal Genomic Fragments Using Affinity Chromatography

Selection or Generation of Molecular Probes

Using the information obtained in Example 1, two UNA-hairpins are prepared, each designed to specifically hybridize to one of the two differentiating alleles. The UNA-hairpins contain an aminohexyl terminus, which is used to covalently attach the UNAs to NHS-activated Sepharose™ 4 Fast Flow beads, using standard protocols (Van Sommeren et al., J. Chromalogr. 639:23, 1993). The two different types of coated beads are then packed into two different affinity columns.

Fragmentation of Genomic DNA

Genomic DNA fragments of about 40kb are prepared by partial digestion with a restriction endonuclease using standard protocols (Sambrook et al., Molecular Cloning: A Laboratory Manual, Cold Spring Harbor Press, N.Y., 1989).

Capture of The DNA on The Affinity Beads

The two affinity columns are connected in series, and the DNA fragments are prepared in buffer and slowly added to the first column, eluted first through the first column, and then through the second column. Excess DNA is slowly eluted out of the end of the second column. The desired maternal and paternal DNA fragments remain bound to the affinity beads. The two columns are separated, and the DNA from each is eluted under denaturing conditions sufficient to disrupt the UNA binding, and recovered for further analysis.

### Example 6

### Isolation of Specific of Maternal or Paternal Genomic Fragments Using Affinity Capillary Electrophoresis

Determination of Heterozygous SNP Site

Using the sample genomic DNA of interest, the genotype is determined at a number of SNP sites on chromosome 3. The genotyping is done using any method, but homogeneous methods such the TAQMAN™ assay (available from PE Biosystems of Foster City, Calif.) and the READIT™ assay (available from Promega of Madison, Wis.) are particularly convenient.

Preparation of The Molecular Probe-Polyacrylamide Conjugate or “Affinity Polymer”

Using the information obtained by the determination of a heterozygous SNP site, an allele specific UNA-hairpin with an aminohexyl terminus is prepared. This oligonucleotide is reacted with N-methacryloyloxysuccinimide to form an acrylamide-UNA conjugate, which is then incorporated into linear polyacrylamide using standard radical initiation conditions to produce an affinity polymer (Anada et al., Electrophoresis 23:2267, 2002).

Preparation of Chromosomal DNA Fragments

Separation of human metaphase chromosomes is performed by flow cytometry (Carrano, et.al., Proc. NatL. Acad. Sci USA 76:1382, 1979 and Langlois, et.al., Proc. Natl. Acad Sci USA 79:7876, 1982). Chromosome 3 is collected and subjected to fragmentation in an agarose plug using the restriction endonuclease Not I (Doggett et al., Nucl. Acids. Res. 20:859, 1992). Fragments sizes of about 1 Mb are produced.

Affinity Capillary Electrophoresis

A capillary column is filled with a dilute solution of the affinity polymer. The agarose plug containing the DNA fragments is melted by heating to 65° C., diluted with buffer, and injected onto the capillary column (Anada et. al., Electrophoresis 23:2267, 2002 and Sudor and Novatny, Electrophoresis 66:2446, 1994). Electric field is applied, and the DNA migrates through the capillary column. The fragment containing the allele of interest is preferentially retarded by the affinity polymer, while all the other DNA fragments migrate at approximately the same (faster) rate. The desired maternal or paternal DNA fragment is collected as it exits the column, and saved for further analysis.

### Example 7

### Haplotyping

The DNA in any of the isolated chromosome or chromosome fragments of Example 2-6 are genotyped by any convenient method, including sequencing. The haplotype is determined directly from the genotype.

### Other Embodiments

Other embodiments of the invention will be apparent to those skilled in the art from a consideration of the specification or practice of the invention disclosed herein. It is intended that the specification and examples provided herein be considered as exemplary only, with the true scope of the invention being indicated by the following claims.

We claim:

1. A method for separating homologous maternal and paternal genetic material comprising steps of:
contacting a sample that includes homologous maternal and paternal genetic material with a first binding entity, wherein the maternal genetic material includes a first allele of a first polymorphic region, the paternal homologue includes a second allele of the first polymorphic region, and the first binding entity includes a first molecular probe that binds preferentially to one of the first or second allele; and
separating the homologous maternal and paternal genetic material by virtue of the preferential binding between the first molecular probe and one of the first or second allele.

2. The method of claim 1, wherein the homologous maternal and paternal genetic material is selected from the group consisting of a pair of homologous chromosomes, fragments of a pair of homologous chromosomes, and polynucleotides that have been derived from a pair of homologous chromosomes.

3. The method of claim 1, wherein the step of contacting is preceded by steps of:
identifying a first polymorphic region that differentiates the homologous maternal and paternal genetic material; and
selecting the first molecular probe based on the step of identifying.

4. The method of claim 3, wherein the step of identifying comprises SNP genotyping a collection of SNP sites within the homologous maternal and paternal genetic material.

5. The method of claim 1, wherein the first molecular probe comprises a polypeptide that includes a DNA-binding motif selected from the group consisting of helix-turn-helix motifs and zinc-finger motifs.

6. The method of claim 5, wherein the polypeptide includes a series of covalently linked zinc-finger motifs.

7. The method of claim 1, wherein the first molecular probe comprises a 9,10-phenanthrenequinone diimine complex of rhodium (III), a 9,10-phenanthrenequinone diimine 2,2′-bipyridyl complex of rhodium (III), or a derivative thereof.

8. The method of claim 1, wherein the first molecular probe comprises a polyamide that has been prepared from amino acids selected from the group consisting of P-alanine, N-methylpyrrole amino acid, N-methylimidazole amino acid, and N-methyl-3-hydroxypyrrole amino acid.

9. The method of claim 8, wherein the polyamide includes two polyamide chains linked via a γ-butyric acid linker.

10. The method of claim 1, wherein the first molecular probe comprises an oligonucleotide.

11. The method of claim 10, wherein the oligonucleotide includes two complementary sequences joined by a linker region.

12. The method of claim 10, wherein the oligonucleotide includes an unstructured nucleic acid, a peptide nucleic acid or a locked nucleic acid.

13. The method of claim 1, wherein in the step of separating the homologous maternal and paternal genetic material, the sample is subjected to electrophoresis.

14. The method of claim 13, wherein the first molecular probe is associated with an electrophoretic tag that alters the electrophoretic mobility of maternal or paternal genetic material that is bound by the first molecular probe.

15. The method of claim 1, wherein the first molecular probe is associated with an affinity tag.

16. The method of claim 15, wherein in the step of separating the homologous maternal and paternal genetic material, the sample is contacted with a solid phase that is associated with a capture agent for the affinity tag.

17. The method of claim 1, wherein the first molecular probe is associated with a solid phase.

18. The method of claim 17, wherein the solid phase is a gel.

19. The method of claim 1, wherein the first molecular probe is associated with a detectable label selected from the group consisting of paramagnetic labels, fluorescent labels, light scattering labels, chemiluminescent labels, absorptive labels, and colorimetric labels.

20. The method of claim 19, wherein the step of separating the homologous maternal and paternal genetic material comprises:
flowing the sample through a flow cytometer;
detecting the presence of one or more labels in the sample; and
separating the homologous maternal and paternal genetic material based on the step of detecting.

21. The method of claim 1 further comprising steps of:
contacting the sample with a second binding entity, wherein the maternal genetic material includes a first allele of a second polymorphic region, the paternal homologue includes a second allele of the second polymorphic region, and the second binding entity includes a second molecular probe that binds preferentially to one of the first or second allele of the second polymorphic region; and
separating the homologous maternal and paternal genetic material by virtue of the preferential binding between the first molecular probe and one of the first or second allele of the first polymorphic region and the preferential binding between the second molecular probe and one of the first or second allele of the second polymorphic region.

22. The method of claim 21, wherein the first molecular probe binds preferentially to an allele within one of the maternal or paternal genetic material and the second molecular probe binds preferentially to an allele in the other.

23. The method of claim 21, wherein the first and second molecular probes both bind preferentially to alleles within the maternal genetic material or both bind preferentially to alleles within the paternal genetic material.

24. The method of claim 21, wherein the sample is contacted with the first and second binding entities sequentially.

25. The method of claim 21, wherein the sample is contacted with the first and second binding entities simultaneously.

26. A method for separating homologous maternal and paternal genetic material comprising steps of:
contacting a sample that includes homologous maternal and paternal genetic material with a first binding entity, wherein the maternal genetic material includes a first allele of a first polymorphic region, the paternal homologue includes a second allele of the first polymorphic region, and the first binding entity includes a first molecular probe that binds preferentially to the first allele and a second molecular probe that binds preferentially to the second allele; and
separating the homologous maternal and paternal genetic material by virtue of the preferential binding between the first and second molecular probes and the first and second alleles, respectively.

27. The method of claim 26, wherein the first molecular probe is associated with a first solid phase and the second molecular probe is associated with a second solid phase, and wherein the step of separating the homologous maternal and paternal genetic material comprises sequentially contacting the sample with the first and second solid phases.

28. The method of claim 26, wherein the first molecular probe is associated with a first solid phase and the second molecular probe is associated with a second solid phase, and wherein the step of separating the homologous maternal and paternal genetic material comprises:
contacting the sample with the first and second solid phases simultaneously; and then
separating the first and second solid phases.

29. The method of claim 26, further comprising contacting the sample with a second binding entity before contacting the sample with the first binding entity, wherein the maternal and paternal genetic material both include a first allele of a second polymorphic region and the second binding entity includes a third molecular probe that binds preferentially to the first allele of the second polymorphic region and a fourth molecular probe that binds preferentially to a second allele of the second polymorphic region.

30. The method of claim 29, wherein the identity of the first polymorphic region that differentiates the homologous maternal and paternal genetic material is unknown at the time that the sample is contacted with the first and second binding entities.

31. A method for separating homologous maternal and paternal genetic material comprising steps of:
passing a sample that includes homologous maternal and paternal genetic material through or over a first binding entity; and
separating the homologous maternal and paternal genetic material by virtue of a difference in the rate at which they pass through or over the first binding entity.

32. The method of claim 31, wherein:
the first binding entity comprises a collection of different families of molecular probes,
each molecular probe within a given family binds reversibly with a specific allele of a polymorphic region that is present within the homologous maternal and paternal genetic material,
no more than one family includes a molecular probe that binds preferentially to an allele of a given polymorphic region, and
the molecular probes within at least one family bind reversibly with an allele that is only present within one of the maternal or paternal genetic material, thereby retarding the passage of maternal and paternal genetic material through or over the first binding entity to different extents.

33. The method of claim 32, wherein:
the different families of molecular probes are associated with one or more solid phases arranged within the same affinity column,
the step of passing comprises passing the sample through said affinity column, and
the maternal and paternal genetic material are retarded to different extents when the sample is passed through said affinity column.

34. The method of claim 32, wherein:
the different families of molecular probes are associated with different solid phases that are arranged within a collection of physically separate affinity columns, and
the step of passing comprises sequentially passing the sample through the collection of affinity columns, and
the maternal and paternal genetic material are retarded to different extents when the sample is sequentially passed through said collection of affinity columns.

35. The method of claim 1 further comprising a step of SNP genotyping at least two SNP sites within the maternal genetic material, the paternal genetic material, or both, after these have been separated.

36. A kit comprising:
a first molecular probe that binds preferentially with an allele of a polymorphic region that is present within a first pair of homologous chromosomes; and
a second molecular probe that binds preferentially with an allele of a polymorphic region that is present within a second pair of homologous chromosomes.

37. A kit for separating homologous maternal and paternal genetic material comprising:
a first molecular probe that binds preferentially with a first allele of a first polymorphic region that is present within the homologous maternal and paternal genetic material; and
a second molecular probe that binds preferentially with a second allele of said first polymorphic region.

38. The kit of claim 37, wherein the first molecular probe is associated with an electrophoretic tag that alters the electrophoretic mobility of maternal or paternal genetic material that is bound by the first molecular probe.

39. The kit of claim 37, wherein the first and second molecular probes are associated with first and second affinity tags, respectively.

40. The kit of claim 39 further comprising:
a first solid phase that is associated with a capture agent for said first affinity tag; and
a second solid phase that is associated with a capture agent for said second affinity tag.

41. The kit of claim 37, wherein the first and second molecular probes are associated with different regions of a solid phase.

42. The kit of claim 41, wherein the solid phase is a gel.

43. The kit of claim 37 further comprising:
a third molecular probe that binds preferentially with a first allele of a second polymorphic region that is present within the homologous maternal and paternal genetic material; and
a fourth molecular probe that binds preferentially with a second allele of said second polymorphic region.

44. The kit of claim 43, wherein the first, second, third, and fourth molecular probes are associated with first, second, third, and fourth solid phases that are arranged within first, second, third, and fourth affinity columns, respectively.

45. A kit for separating homologous maternal and paternal genetic material comprising a collection of different families of molecular probes, wherein each molecular probe within a given family binds reversibly with a specific allele of a polymorphic region that is present within the homologous maternal and paternal genetic material, and no more than one family includes a molecular probe that binds preferentially to an allele of a given polymorphic region.

46. The kit of claim 45, wherein the molecular probes are associated with one or more solid phases arranged within one or more affinity columns.
